(19) United States
(12) Patent Application Publication
(10) Pub. No.: US 2025/0257300 A1
(43) Pub. Date: Aug. 14, 2025

(54) MICROTITER-PLATE-BASED HIGH THROUGHPUT PERFUSION BIOREACTOR

(71) Applicant: THE GEORGE WASHINGTON UNIVERSITY, Washington, DC (US)

(72) Inventors: David MCLEOD, Arlington, VA (US); Weizhen LI, Washington, DC (US); Lai WEl, Baltimore, MD (US); Matthew W. KAY, Bethesda, MD (US); Emilia ENTCHEVA, Washington, DC (US); Zhenyu Ll, Mclean, VA (US)

(21) Appl. No.: 19/177,137

(22) Filed: Apr. 11, 2025

Related U.S. Application Data
(63) Continuation-in-part of application No. PCT/US2023/076703, filed on Oct. 12, 2023.
(60) Provisional application No. 63/415,507, filed on Oct. 12, 2022.

(51) Int. Cl. C12M 1/32; C12M 1/00; C12M 1/34

(52) U.S. Cl. CPC C12M 23/12; C12M 23/22; C12M 29/10; C12M 41/00; C12M 41/12; C12M 41/34

(57) ABSTRACT

A reactor for use with a media and a standard microtiter well plate having a top surface and a well with a side wall, a bottom portion, and a transparent bottom, and a target substance at the bottom of the well. The reactor has a support layer with a lower surface that extends substantially parallel to the top surface of the well plate. A perfusion plug extends outward from the lower surface of said support layer. The perfusion plug has a distal end that is at a distance from the bottom of the well to form a bottom flow channel at the bottom portion of the well. An inlet channel extends through the support layer and through or around the perfusion plug. The inlet channel is in fluid communication with the bottom flow channel. An outlet channel extends through said support layer and through or around the perfusion plug, the outlet channel in fluid communication with the bottom flow channel. The media can travel through the inlet channel to the bottom flow channel to contact the target substance, then through the outlet channel to exit the reactor.

### CROSS-REFERENCE TO RELATED APPLICATIONS

This application is a continuation-in-part of PCT/US2023/076703, filed Oct. 12, 2023, which claims the benefit of priority of U.S. Provisional Application No. 63/415,507 filed on Oct. 12, 2022, the content of which is relied upon and incorporated herein by reference in its entirety.

### GOVERNMENT LICENSE RIGHTS

This invention was made with government support under NSF Grant No. CBET 1743662; NSF Grant No. EFMA 1830941; and NIH Grant No. R01HL144157. The U.S. government has certain rights in the invention.

### BACKGROUND

In basic biomedical research and practical applications, it is desirable to maintain cells and tissues under conditions like those in in-vivo environments including oxygenation, fuel and nutrient delivery and waste removal etc. However, current gold standard microtiter plates (e.g. 96 wells microplates) lacks continuous perfusion capabilities to precisely control these conditions. Large-scale bioreactors used in biomanufacturing can provide perfusion or mixing but they are complex, bulky and expensive. Here, a bioreactor refers to any device, apparatus or system that supports a biologically active environment. It is highly desirable to have a self-contained perfusion bioreactor system compatible with standard microtiter plates (e.g., 96-well microplates), which is also compact enough to be placed in a standard incubator or a microscope cage incubator or as a portable system. Such microtiter plate-based perfusion bioreactors can be used for basic research such as cell/tissue slice/spheroid/organoid culturing, drug screening and toxicity testing, disease modeling, biofilm growth and study, antibiotic susceptibility testing, and biomanufacturing.

Biofilms manifest as microbial clusters adherent to a given surface and enclosed within a self-produced matrix that resists environmental stresses. This resistance presents a particular danger for medical devices as up to 60% of hospital-acquired infections are associated with biofilm infections of implantable devices (Bryers 2008). Studies have observed differences in biofilm structure and gene expression based on the presence of many environmental stresses including hypoxia and shear stress, as well as the presence of multiple strains of bacteria. The classic phenotypic understanding of biofilm formation involves the transition from planktonic to sessile lifestyle through surface attachment and mediated by a variety of genetic and environmental signals that vary from species to species. One major difference between biofilms and planktonic cells is susceptibility to antimicrobial agents. For example, one study shows that Pseudomonas aeruginosa biofilms growing on urinary catheters are 1000 times more resistant to tobramycin than planktonic cells (Nickel 1985). Several known mechanisms of antibiotic resistance provided by biofilms include extracellular polymeric substances, extracellular DNA, oxidative stress response, efflux pumps, and horizontal gene transfer. Individually, no single mechanism accounts for this heightened antibiotic recalcitrance, but understanding the biofilm-specific mechanisms for antibiotic resistance and tolerance could lead to more effective antimicrobial therapies to treat or prevent infections.

Current methods for culturing biofilms involve either static or flow conditions. Static biofilm reactors have the advantages of simple design and ease of use. 96 well-based static reactors standardize fluid handling and provide compatibility with high-throughput robotic systems. A major drawback of these devices is the tendency for planktonic bacteria to accumulate on the bottom of the well due to gravitational settling and contaminate the sample. Designs such as the Calgary device modify the growth surface through the insertion of hanging pegs into the wells of the microtiter plate (Ceri 1999). Directing biofilm growth to the pegs eliminates potential contaminants by allowing them to accumulate at the well bottom, but these devices have the drawback of requiring the removal of the cover periodically to analyze the biofilm and therefore, these devices are not compatible with in situ measurements. As used here, in situ measurements refers to making measurements on the biofilm when it is in the well without removing it, and without removing the cover (which loses control of the growth environment since the gases would be exchanged when the cover is removed). This can allow real-time monitoring of biofilm growth, it's dynamic response to antibiotic treatments etc.

Culturing under flow, while more complicated, inherently removes contamination from planktonic bacteria and debris by pushing these contaminants downstream. Furthermore, flow has been shown to alter biofilm behavior by imposing conditions such as shear stress, oxygen delivery and nutrient availability (Nickel 1985). Recently, culturing platforms have incorporated flow into their designs to better mimic physiological conditions such as those in urinary catheters and explore relationships between flow, phenotype, and genotype. Along with the inclusion of flow, recent reactors have looked to quantify the dynamics of biofilm growth more rigorously with a focus on in situ characterization as to not disturb these dynamics.

One such reactor termed BioFlux integrates pneumatically controlled perfusion within a 96 well format with microscopy to provide in situ analysis. This reactor has been used to study biofilm formation and growth potential in situ (Abberton 2016) but requires the use of a proprietary, modified microtiter plate. Furthermore, as the biofilm is grown in a channel between wells, this plate is incompatible with techniques that rely on the standard 96 well format such as a plate reader. Other modalities include an impedance-based platform with excellent temporal resolution to investigate the initial instability of bacterial adhesion (Wang 2020). This method demonstrates high-throughput through the use of a microtiter plate as well as in situ optical analysis by confocal laser scanning microscopy. However, this method lacks continuous perfusion, instead relying on shaking to provide shear. Farid et al. demonstrated a platform using optical coherence tomography (OCT) to monitor biofilm growth in situ on fabricated graphene oxide and PVDF membranes. OCT measures biofilm thickness and morphology and therefore, this technique shows great promise for in situ characterization in real time that could incorporate three-dimensional biofilm growth but is limited by its throughput for individual membranes. In summary, these reactors either lack perfusion capabilities, lack compatibility with a standard high-throughput culturing platform such as a 96 well plate, or lack compatibility with in situ optical analysis.

Several micro perfusion solutions have been proposed and applied to non-bacterial dynamic cell culture (Chen 2011; Goral 2013; Yoshimitsu 2014; Kim 2015; Parrish 2018). However, these designs increase complexity by requiring a custom microtiter plate. Previously, our lab has demonstrated a high-throughput microfluidic perfusion culturing technology compatible with standard 96 well plate termed HT-uUPS (Wei, 2017). This cover was fabricated using similar techniques to this work but from a soft, gas-permeable material PDMS instead of acrylic and for use in culturing human induced pluripotent stem-cell-derived cardiomyocytes (iPSC-CM) and engineered excitable (spiking HEK) cells.

In basic biomedical research and drug screening applications, it is desirable to maintain cells and tissues under conditions like those in in-vivo environments including oxygenation, fuel and nutrient delivery and waste removal etc. However, current gold standard microplates (e.g., 96 wells) lacks continuous perfusion capabilities to precisely control these conditions.

In addition, it is noted that oxygen is important for nearly all biological processes. The function of aerobic cells relying on oxidative phosphorylation, such as cardiomyocytes, is highly dependent upon oxygen availability. Precise monitoring of oxygen levels in the immediate cell vicinity (peri-cellular oxygen) will provide invaluable insight into the metabolic state of the cells. Over the last two decades, human induced pluripotent stem cell derived cardiomyocytes (hiPSC-CMs) have emerged as a new scalable experimental model for cardiovascular research and translation. The growth of these cells in vitro is being optimized for applications in regenerative medicine, drug development, cardiotoxicity testing, and other personalized medicine applications. iPSC-CM oxygen consumption and metabolism are considered of key importance for their maturity.

For drug development and cardiotoxicity studies, human iPSC-CMs are typically grown in static culture using glass-bottom high-throughput format plates (96-well or 384-well plates). In such conditions, the only oxygen diffusion path is from the top, through the solution. Long-term studies of peri-cellular oxygen dynamics in human cardiac cells in such high-throughput plates are lacking, yet highly desirable.

Historically, oxygen sensing has evolved, starting with amperometric measurements using a Clark electrode, where the electrochemical reduction of oxygen is registered by changes in electric current. This method still serves as the gold standard. Yet, it has limitations, especially for assessing oxygen dynamics in small spaces/volumes, including assessment of peri-cellular oxygen. This is due to the consumption/depletion of oxygen at the sensor during the measurement and the general difficulty in miniaturizing this type of sensor. Contactless optical methods present an alternative. A variety of fluorescent dyes have been developed to register very low (<5%) oxygen levels, including near-infrared indicators for in vivo measurements. The limitations of such dyes include difficulty in providing a quantitative assessment and in covering a broader range of physiological values.

The Seahorse XF platform is a high-throughput version of an optical oxygen measurement system. It has numerous applications in rigorous metabolic profiling of mammalian cells and isolated mitochondria, including iPSC-CMs. It provides quantitative assessments of oxygen consumption rate (OCR) and extracellular acidification rate (ECAR), including in a 96-well format, as it offers oxygen and pH measurements through the optical sensors embedded in the tips of the fiber optics array. However, the Seahorse assay is applicable only to acute terminal measurements and therefore cannot be used for long-term tracking of peri-cellular oxygen dynamics.

Luminescence-based oxygen sensors, often combined with fiber optics, have been demonstrated to offer reliable oxygen tracking over time. Their operation is based on dynamic oxygen quenching of fluorescence, as reflected in the Stern-Volmer relationship between oxygen concentration and fluorescence. Quantitative optical oxygen sensing is typically done through life-time measurements (using frequency modulation) or through ratiometric intensity measurements with an oxygen-responsive dye (e.g., ruthenium-based) and a reference dye). Scalability with such luminescence-based oxygen sensors has been achieved through improved matrix embedding of the dye and production of oxygen-sensing scaffolds for space-resolved measurements, as well as through advances in visualization with high spatiotemporal resolution.

### SUMMARY

The present disclosure is for a high-throughput microfluidic perfusion biofilm reactor (HT-uPBR) compatible with a standard 96-well microtiter plate and in situ optical evaluation techniques. The system is demonstrated for large-scale culture under controlled shear stress for prolonged periods within a standard incubator. The system was validated by applying it to E. coli biofilms and evaluating biomass and viability after 24 hours with a fluorescence microscope and standard microplate reader.

Biofilm infections represent a major public health threat due to their high tolerance to antimicrobials and the lack of specific anti-biofilm drugs. To develop such drugs, it is crucial to have high-throughput biofilm growth systems that can emulate in vivo conditions without the cost and complexity of animal models. However, no current biofilm reactor can provide in vivo-like conditions in a high throughput standard microtiter format. This disclosure demonstrates a novel high-throughput (HT) microfluidic perfusion biofilm reactor (HT-uPBR) compatible with a standard 96-well microtiter plate for in situ optical analysis. A snap-on liquid-tight cover for standard microtiter plates was designed and fabricated with fluidic channels to provide closed-loop recirculating perfusion.

The system provides in vivo-like conditions with controlled shear stress and nutrient delivery. The disclosure describes the system fabrication and usage in optical analysis of biomass and viability of Escherichia coli (E. coli) biofilms. The HT-uPBR was set to perfuse at 1 mL/min corresponding to an average shear rate of approximately 5.7 s⁻¹ on the bottom surface of a single well. Biofilms are detected on well plate bottoms and measured using a fluorescence microscope and plate reader to determine biomass and viability. Samples cultured in the HT-uPBR showed increased biomass while maintaining viability after 24 hours. The HT-uPBR can further be combined with HT antibiotic susceptibility testing and additional optical techniques such as time-lapse imaging to improve understanding of the drug reaction mechanism as well as the optimization of drug combinations and delivery profiles.

In addition, the system provides a high-throughput platform for longitudinal optical sensing of peri-cellular oxygen in human iPSC-CMs and human cardiac fibroblasts in 96-well format within a standard cell culture incubator. The system is based on the VisiSensTD oxygen imaging system (PreSens Precision Sensing GmbH, Germany) and our high throughput microfluidics-based uninterrupted cell culture perfusion system (HT-uUPS). Results demonstrate that the system provides accurate and reproducible long-term measurements of peri-cellular oxygen levels that is valuable for studies of cellular oxygen consumption, metabolic perturbations, and characterization of the maturation of cultured iPSC-CMs.

This summary is not intended to identify all essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

In this disclosure a complete self-contained microfluidic perfusion system is provided which is compatible with standard microtiter plates (e.g., 96-well microplates), which is also compact enough to be placed in a standard incubator or a microscope cage incubator or as a portable system. Such microtiter plate-based perfusion bioreactors can be used for basic research such as biofilm/cell/tissue slice/spheroid/organoid culturing (e.g., cardiomyocyte culturing), drug screening and toxicity testing, disease modeling, biofilm growth and study (e.g., David McLeod, Lai Wei & Zhenyu Li, A standard 96-well based high throughput microfluidic perfusion biofilm reactor for in situ optical analysis, Biomedical Microdevices volume 25, Article number: 26 (2023), which is incorporated herein by reference), antibiotic susceptibility testing, and biomanufacturing. Microtiter plates, also called microplates are standard tools in biomedical research and clinical testing with well numbers ranging from 6 to 1536 per plate, and plates with well volumes ranging from 5 microliters (1536 well) to 5 milliliters (6 well)). https://en.wikipedia.org/wiki/Microplate; see Li Weizhen, McLeod David, Ketzenberger John T., Kowalik Grant, Russo Rebekah, Li Zhenyu, Kay Matthew W., Entcheva Emilia, High-throughput optical sensing of peri-cellular oxygen in cardiac cells: system characterization, calibration, and testing, Frontiers in Bioengineering and Biotechnology, Vol. 11, 2023, DOI: 10.3389/fbioe.2023.1214493, whereby all documents mentioned throughout this disclosure are incorporated herein by reference.

### BRIEF DESCRIPTION OF THE FIGURES

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIG. 1(a) is a perspective view of the microfluidic perfusion cover for a standard 96-well microtiter plate and of the microfluidic plate cover with channels 1 mm×0.5 mm×9 mm;

FIG. 1(b) is an enlarged cross-sectional view taken along the box X of FIG. 1(a), a 2D Schematic side illustration of the microfluidic plate cover;

FIG. 1(c) is a full system schematic, diagram of system components including a media reservoir, piezoelectric pump, cover assembly, flow rate sensor, and electronics, and a real picture of the system;

FIG. 2(a) is a schematic side view showing the uniform shear design;

FIG. 2(b) is a 3D cut-through view of a perfusion cover inserted into a microtiter plate, with only two wells in the plate shown;

FIG. 2(c) is a schematic side view showing another uniform shear design;

FIGS. 3(a)-(d) show an example uniform shear cover on a 96-well plate (a serial flow path is used in this example);

FIG. 3(a) is an assembled perfusion cover on a standard 96-well plate;

FIG. 3(b) is an exploded view of a perfusion cover and a PDMS gasket on a standard 96-well plate;

FIG. 3(c) is an exploded cut-through view of a perfusion cover on a microtiter plate, with only two wells in the plate shown;

FIG. 3(d) is a side view of a perfusion cover on a standard 96-well plate;

FIG. 4(a) shows Computation Fluid Dynamics (CFD) simulations to determine optimal design parameters for managing fluid shear stress applied to the well bottom with nearly uniform flow at the well bottom;

FIG. 4(b) shows shear stress at the well bottom;

FIG. 4(c) shows average shear stress for various lengths of the perfusion plug fitting;

FIG. 5 is a diagram showing percentage of fully developed flow (a measure of uniformity) at the well bottom as a function of liquid column height (i.e. cover insert depth);

FIGS. 6(a), 6(b) are diagrams showing shear stress magnitude can be controlled by changing the liquid column height (i.e., cover insert depth);

FIG. 7 is a serial flow path structure within the perfusion cover;

FIG. 8 is a parallel flow path structure within the perfusion cover;

FIG. 9 is a concentration gradient generator integrated in the perfusion cover; where the two inputs can be culture media with two different oxygen concentrations or with two different drug concentrations;

FIG. 10 is a block diagram showing threshold-based close-loop flow rate control;

FIG. 11 shows a built-in temperature sensor on the perfusion cover insert and a TEC heater/cooler below the microtiter plate can be used for closed-loop temperature control of the perfused well;

FIGS. 12(a)-12(e) show a system and workflow for high-throughput optical measurements of peri-cellular oxygen;

FIG. 12(a) shows an incubator-deployed imaging system for 96-well plates, with an RGB camera, ring LED illuminator and bellows extension to fit a 96-well plate; the inset shows top-down view with the lens and the LED ring with excitation filter;

FIG. 12(b) is a schematic of a single well with an optical oxygen sensor composition and placement in half of the well; peri-cellular oxygen is imaged ratiometrically through the glass bottom of the well plate; partial coverage allows for other parameters to be measured optically, e.g. voltage, calcium etc;

FIG. 12(c) shows laser-cutting of semicircular oxygen sensor patches, 5 mm diameter;

FIG. 12(d) shows mounting of the oxygen sensors: patches are detached from the adhesive backing and placed in wells using vacuum tubing;

FIG. 12(e) are images of the oxygen sensors in wells, raw optical readout and processed readings of oxygen; in the example, the upper left quadrant of the plate has been treated with oxygen-depleting Na₂SO₃;

FIGS. 13(a)-13(c) show spectral characterization of the optical system for peri-cellular oxygen imaging and confirmation of an inverse Stern-Volmer relationship;

FIG. 13(a) is a schematic of the setup used to characterize oxygen-sensitive responses in a beaker of water;

FIG. 13(b) shows spectral results in water: excitation light emission is at 409 nm; oxygen-sensitive emission peak is at 653 nm, with emission decreasing as O₂ concentration increases;

FIG. 13(c) is a schematic and photo of the setup for spectral characterization of oxygen responses in a 96-well microplate with a specialized microfluidic cover (our HT-uUPS system);

the oxygen sensor pad is at the bottom of one well;

FIG. 13(d) shows spectral data in cell culture media, showing oxygen-dependent spectral shifts at 653 nm, consistent with results from water shown in FIG. 13(b);

FIG. 13(e) is an inverse Stern-Volmer relationship was derived from the ratios (intensity at 653/intensity at 510), measured from the spectra shown in panel (D), which is linear for the low oxygen range considered (<20%);

FIGS. 14(a), (b) show issues with Version 1 (FIG. 1(b) design, FIG. 14(a): Non-uniform bottom shear stress. Shear stress is highest at center but quickly decreases in every radial direction from the center, FIG. 14(b): Priming the system can be an issue. Version 1 requires pre-filling the wells with media before applying the cover because the geometry of the cover traps air present within the wells;

FIG. 15(a) version 1 (FIG. 1(b)): simulated ide flow velocity (top left), simulated bottom shear stress (bottom left), plotted velocity profile at center (top right), priming of the cover (bottom right);

FIG. 15(b) version 2 (FIG. 2(a)): simulated ide flow velocity (top left), simulated bottom shear stress (bottom left), plotted velocity profile at center (top right), priming of the cover (bottom right). Version 2 shows improved bottom shear stress uniformity and priming over Version 1; and

FIG. 16(a), (b) CAD overview of the design for 4×8 Version 2 cover with varying liquid column heights, FIG. 16(a) Isometric view of assembly, FIG. 16(b) Side view showing component layers and how different shear stress conditions may be applied serially.

The figures show illustrative embodiment(s) of the present disclosure. Other embodiments can have components of different scale. Like numbers used in the figures may be used to refer to like components. However, the use of a number to refer to a component or step in a given figure has a same structure or function when used in another figure labeled with the same number, except as otherwise noted.

### DETAILED DESCRIPTION

In describing the illustrative, non-limiting embodiments illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments are described for illustrative purposes, it being understood that the description and claims are not limited to the illustrated embodiments and other embodiments not specifically shown in the drawings may also be within the scope of this disclosure. The Applicant hereby incorporates by reference in its entirety, High-Throughput Microfluidic Perfusion Bioreactor Compatible with Standard 96 Well Plate, Dissertation by David McLeod, May 19, 2024.

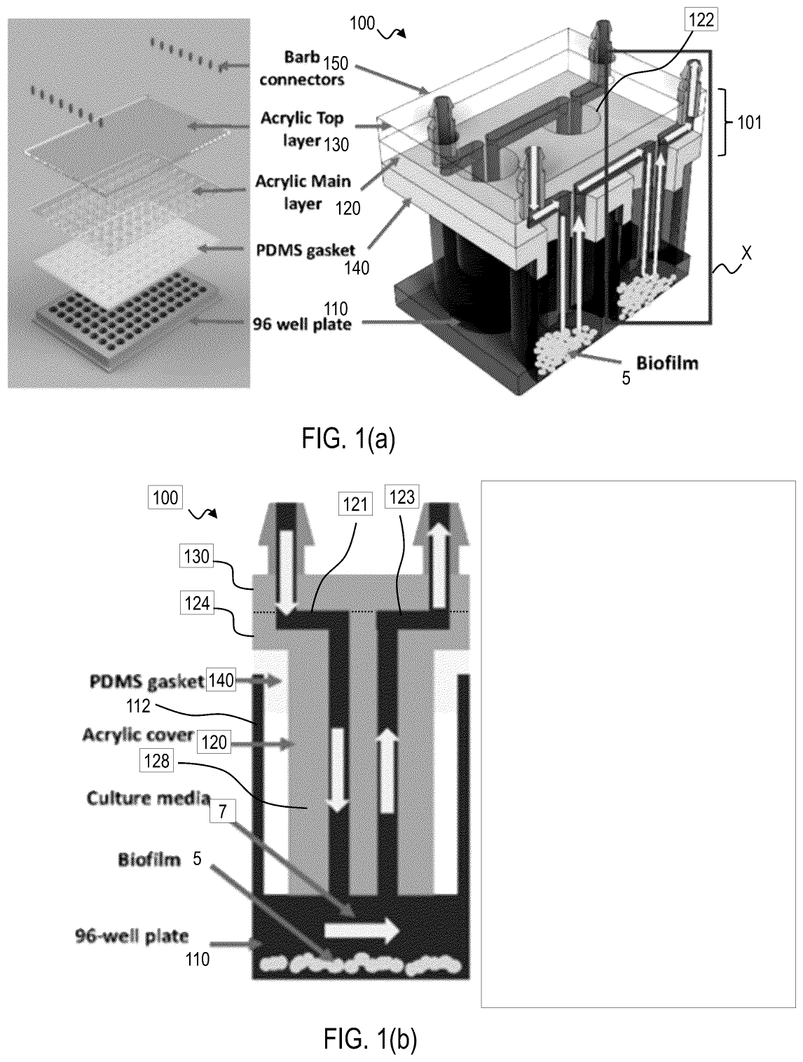

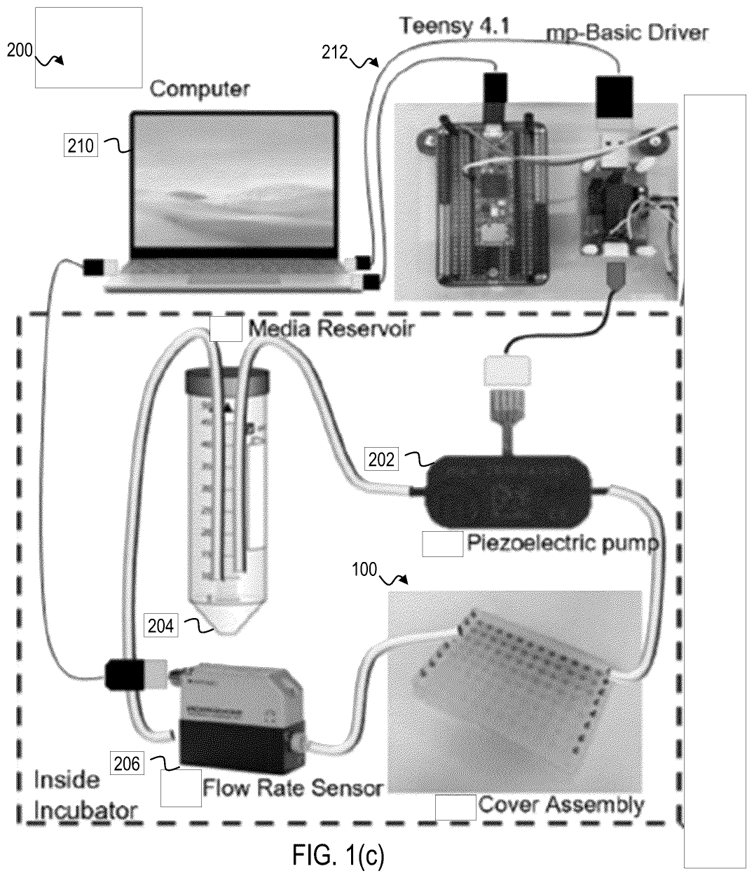

Turning to the drawings, FIGS. 1-3 show a microtiter-plate-based high throughput perfusion bioreactor system or apparatus 100 in accordance with an example non-limiting embodiment of the disclosure. Referring to FIGS. 1(a), 1(b), the apparatus 100 generally has a well plate 110; a cover 101 formed by a main layer 120, top layer 130, and gasket 140; and one or more connectors 150.

The multichannel microfluidic plate cover 101 enables the introduction of continuous shear stress and high throughput perfusion that promotes biofilm generation in a standard 96-well microplate 110. The multichannel microfluidic plate cover 101 includes gas impermeable the two-layered structure made of acrylic Polymethyl methacrylate (PMMA) or polystyrene or cyclic olefin copolymer (COC) 120, 130 and the soft gasket 140 made of polydimethylsiloxane (PDMS). Those hard plastics are compatible with industrial injection molding manufacturing, though other materials can be utilized, including soft or flexible/bendable materials that are impermeable to gases and liquid (i.e., gas tight and liquid tight).

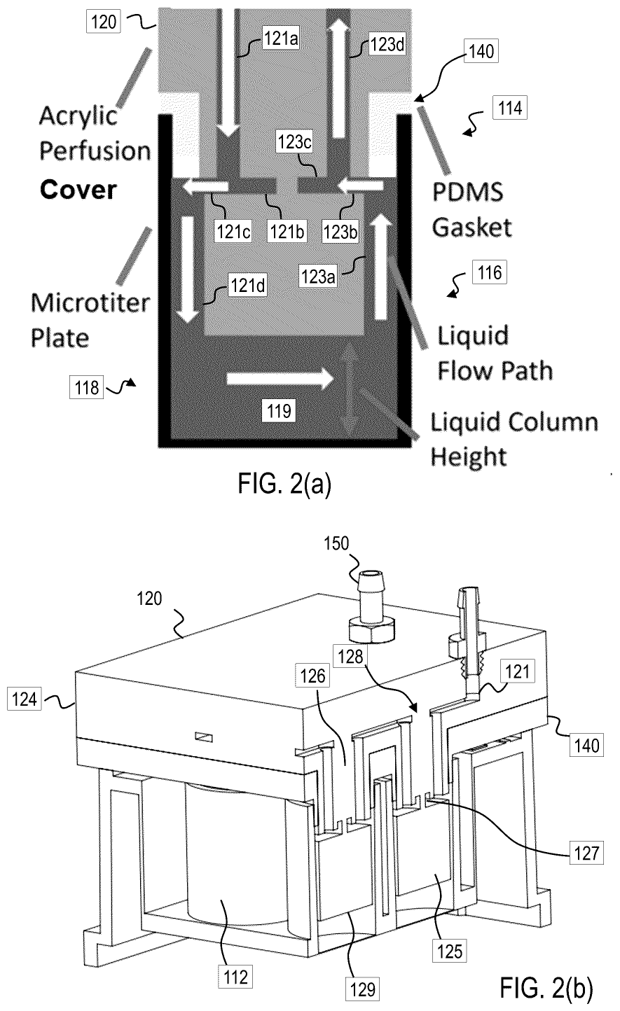

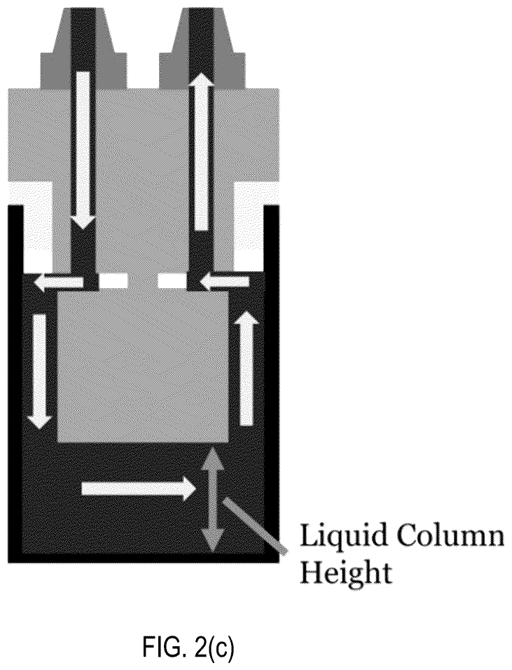

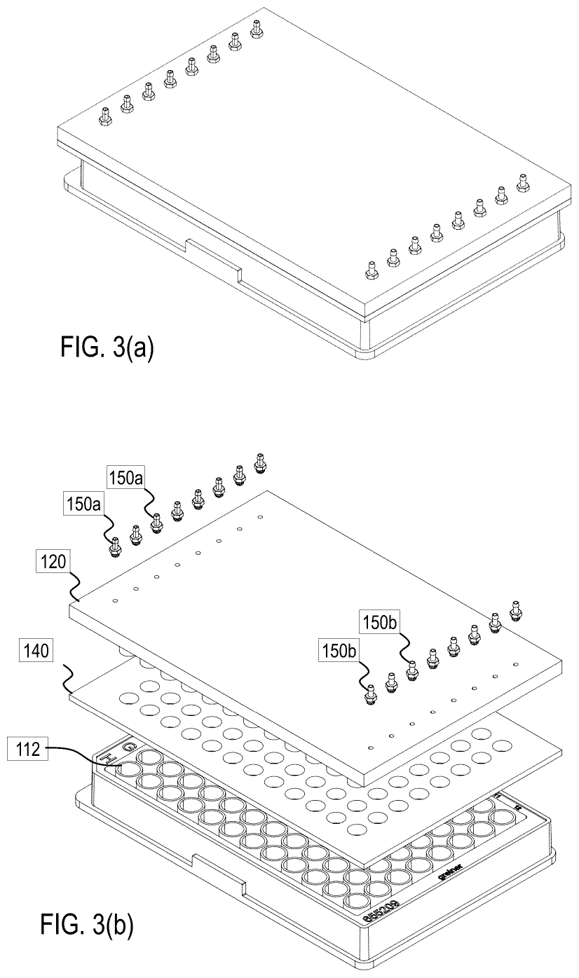

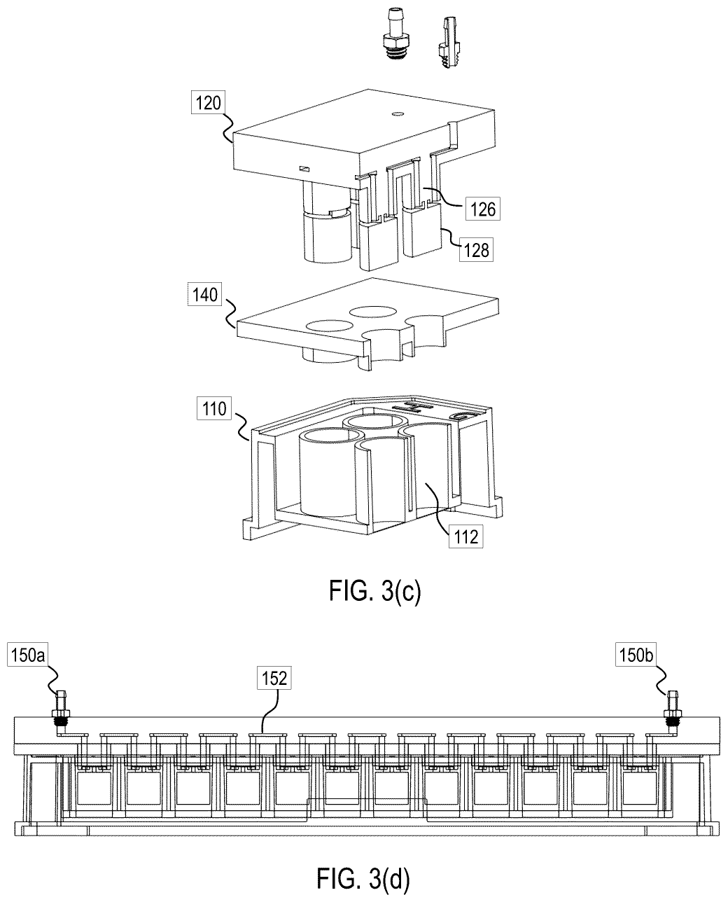

The acrylic main layer 120 has fluidic channels 121, 123 on the top side connecting each well, enabling serial perfusion. These channels measure 1 mm in width, 0.5 mm in height, and 9 mm in length. Cylindrical-shaped perfusion plugs 128 on the other side of the acrylic main layer can be inserted into standard 96-well microplates 110 and set the distance from the cover to the bottom of the well which, along with the volumetric flow rate, determines the shear stress experienced by the cells growing on the bottom of the well. Two channels 121, 123 measure 0.5 mm in diameter and are located at the center of each plug structure, 1.5 mm away from each other, enabling media to get in and out of each standard 96-well microplate's well. The size of the cover 101, channels 121, 123, and perfusion plug 128 height (i.e., length) can vary based on the requirements of different experiments. The top layer also has multiple threaded holes close to its edge, enabling the connection of multiple barb-to-threaded connectors 150, simplifying the tube connection and system setup. As best shown in FIGS. 2(b), 3(c), the connectors 150 can have a barbed or threaded end that is press-fit or screwed into the respective channel 121, 123, and a barbed end that couples to a tubing that can lead to a pump, flow rate sensor, media reservoir, etc. (FIG. 1(c)).

The well plate 110 can be, for example, a standard well-plate having multiple wells 112, here shown as 96 wells 112. That is, the cover 101 is designed to work with a “standard” well plate 110, which as used here has one or more wells 112 that are typically closely arranged in rows and columns. Other commercially available 96-well format bioreactor systems such as Predict96 (Draper) and BioFlux (Fluxion) require significant modification of the well-plate to achieve perfusion, for example, 96-well microplates used in such systems are either custom made, or the bottom of a standard microplate is removed and a custom bottom with perfusion structures is glued to the original standard microplate. But these modifications make the plates incompatible with common optical analysis techniques such as a plate reader, for example, the custom-made microplates have either non-transparent windows or complex fluidic structures that will block in situ plate reader-based measurements. In addition, some custom-made microplates no longer have the standard dimensions so that they cannot be inserted into plate readers. These modifications also increase the cost and size of the systems due to the need for specialized pneumatics and integrated imaging. In contrast, the system 200 and cover 101 require no modification to the 96 well plate to achieve high throughput, customizable flow conditions and flow is achieved through a miniature piezoelectric pump. The well plate 110 need not be specially configured for use with the cover 101, and need not contain any specialized components or features.

As best shown in FIGS. 1(b), 2(a), 2(b), each well 112 of the standard well plate 110 is cylindrical in shape with a general U-shaped cross-section. The well 112 has a closed bottom and an open top, and can a single side (circular) or multiple side walls. Referring to FIG. 2(a), the well 112 has a top portion 114, middle portion 116, and bottom portion 118. As further shown in FIG. 1(b), a material such as biofilm 5 is located at the bottom portion 118 at the bottom of the well 112. A culture media 7 flows through the main layer 120 of the well plate 110 and into and through the bottom portion of the well 112, where the media 7 comes into contact with the biofilm 5. The top surface of the well plate 110 is substantially planar and the well openings can be accessed at the top of the well plate 110.

The main layer 120 can be configured in a number of different architectures. For example, as shown in FIG. 1(b), the main layer 120 has an upper support portion 124 and a lower main body portion, here referred to as a perfusion plug 128. The support portion 124 forms a flat square or rectangular layer that extends parallel to and horizontally across the planar top surface of the well plate 110. The main layer 120 can be formed as a single integral member with an inlet channel 121 and outlet channel 123 formed therein. However, in the embodiment of FIGS. 1(a), 1(b), a top layer 130 is placed on the top surface of the main layer 120 to facilitate manufacture of the channels 121, 123. That is, the horizontal sections of the channels 121, 123 can be formed in the top surface of the main layer 120, then the top layer 130 can be placed over the top surface of the main layer 120 and adhered thereto.

The perfusion plug 128 has a cylindrical shape to match the shape of the well 112, and is slightly smaller than the well 112. The perfusion plug 128 extends downward from the lower or bottom (i.e., bottom facing) surface of the support portion 124. As illustrated, any number of perfusion plug 128 can be provided. In the embodiment shown, there are 96 main body portions 128, one for each well 112 of the well plate 110.

As further illustrated in FIG. 1(b), each of the perfusion plug 128 is aligned with a respective one of the wells 112, and extends downward into the interior of the respective one of the wells 112. The perfusion plug 128 has a flat bottom and an outer side circumference. The outer side of the perfusion plug 128 is straight and parallel to the inner wall surface of the well 112.

The perfusion plug 128 is solid, with the inlet channel 121 and the outlet channel 123 formed therein. The channels 121, 123 extend completely through the support portion 124 and the top layer 130 (in the embodiment where a separate top layer 130 is provided) and longitudinally through the perfusion plug 128. Thus, the inlet channel 121 forms an intake opening at the top surface of the support portion 124 (or the top surface of the top layer 130), and an exit opening in the bottom of the perfusion plug 128. And, the outlet channel 123 forms an intake opening at the bottom of the perfusion plug 128, and an exit opening at the top surface of the support portion 124. The openings can be curved, to facilitate travel of the media 7. In one embodiment, the main layer 120 and the top layer 130 are made of a hard plastic, such as acrylic. Accordingly, the channels 121, 123 extend vertically through the top layer 130, then horizontally at the main layer 120, and again vertically through the main layer 120 and the perfusion plug 128. In other embodiments, the horizontal portion is not provided.

The gasket 140 is best shown in FIGS. 1(a), 1(b), 3(c). The gasket 140 is a flat thin layer that is placed between the main layer 120 and the wells 112 to form a liquid tight seal therebetween. The gasket 140 can have openings 122 aligned with the perfusion plug 128 of the main layer that are slightly smaller than the diameter of the perfusion plug 128, so that the gasket 140 forms a friction fit about the perfusion plug 128 and the gasket 140 can bend downward against the inside surface of the well 112. Or the gasket 140 can be pre-formed to have an inverted L-shape so that it forms a seal at the top opening and top portion of the well 112 and the perfusion plug 128 and/or support portion 124 of the main layer 120. The gasket 140 can be made of a flexible material, such as rubber or PDMS. As shown (FIG. 1(a)), the channels 121, 123 extend through the openings 122 in the gasket 140. And in other embodiments, the channels can be separate or discrete elements (such as tubes with side walls and a circular, square or rectangular cross section) that are received in openings.

The soft gasket 140 provides sealing between the standard 96-well microplate 110 and the microfluidic plate cover 101. The soft gasket has 96 O-ring-like structures whose outer diameter is approximately 0.2 mm larger than the inner diameter of each well on a standard 96-well microplate. It is noted that the inner well diameter of 96-well plates are actually not standardized so the gasket design can be tailored to a given microplate 110. The inner diameter matches the diameter of the perfusion plugs on the acrylic layer, as shown in FIGS. 1(a), 1(b). The elastomeric material used for the soft gasket enables the slightly larger gasket to deform and snap into each well in standard 96-well microplates and create a snap fit with an air-tight and liquid-tight sealing. A mixture of two different silicone elastomers, Sylgard 184, which provides the rigidity needed, and Dragon Skin, which improves the tear strength, with a 1:2 ratio, enhances the durability and sealing of the soft gasket. The heights and diameters of the O-ring-like structure can vary based on the size of the specific 96-well microplate and the desired backpressure tolerance. The apparatus sealing can also be improved by applying force on the top of the cover, which can be achieved using a clamp or weight.

In one embodiment, the gasket 140 can be a Polydimethylsiloxane (PDMS) (Sylgard 184) and high-tear strength silicone rubber (Dragon Skin 10 Fast) from Ellsworth and Smooth-on respectively. The main layer 120 and top layer 130 are acrylic sheets (12″×12″×½″ and ¼″ clear scratch- and UV-resistant cast acrylic sheet, part #: 8560K354). The acrylic cover 101 and PDMS gasket 140 were assembled to a standard, non-treated 96 well plate (Cell Treat 229596) and connected to other system components by 1/32″ inner diameter peroxide-cured silicone tubing (Cole-Parmer SK-95866-00).

Referring to FIG. 1(c), an illustrative example of the full perfusion system 200 is shown having electronic, software, and fluidic components. The system 200 includes one or more media reservoirs 204, one or more pumps 202 (e.g., piezoelectric pump), cover assembly 100, and an optional flow rate sensor 206 and/or other optional sensors (O2, pH, Temperature, glucose, lactate etc.), a controller 210 such as a computer for electronic control and software subsystems, and a driver 212. As illustrated, the pump 202 has a first end that is connected by a first tubing to an inlet or outlet connector 150 of the cover assembly 100. The second opposite pump end is connected by a second tubing to a media reservoir 204, which contains media for circulation through the cover assembly 100. The flow rate sensor 206 is connected by a third tubing to the media reservoir, and is also connected by a fourth tubing to the inlet or outlet connector 150 of the cover assembly 100.

If the pump 202 is connected to an inlet connector 150a (see FIG. 3(d)) and the sensor 206 is connected to the outlet connector 150b, then the pump pulls media from the media reservoir 204 through the first and second tubing to the cover assembly 100, then through the fourth tubing to the flow rate sensor 206 and then through the third tubing back to the media reservoir 204. If the pump 202 is connected to an outlet connector 150b and the sensor 206 is connected to the inlet connector 150a, then the pump 202 pulls media from the media reservoir 204 through the third tubing to the flow rate sensor 206, then through the fourth tubing to the cover assembly 100, and then through the first tubing to the pump 202 and the second tubing to the media reservoir 204. Of course, the components can be arranged in any suitable manner, such as that the flow rate sensor 206 and pump 202 can be directly connected between the media reservoir 204 and the cover assembly 100.

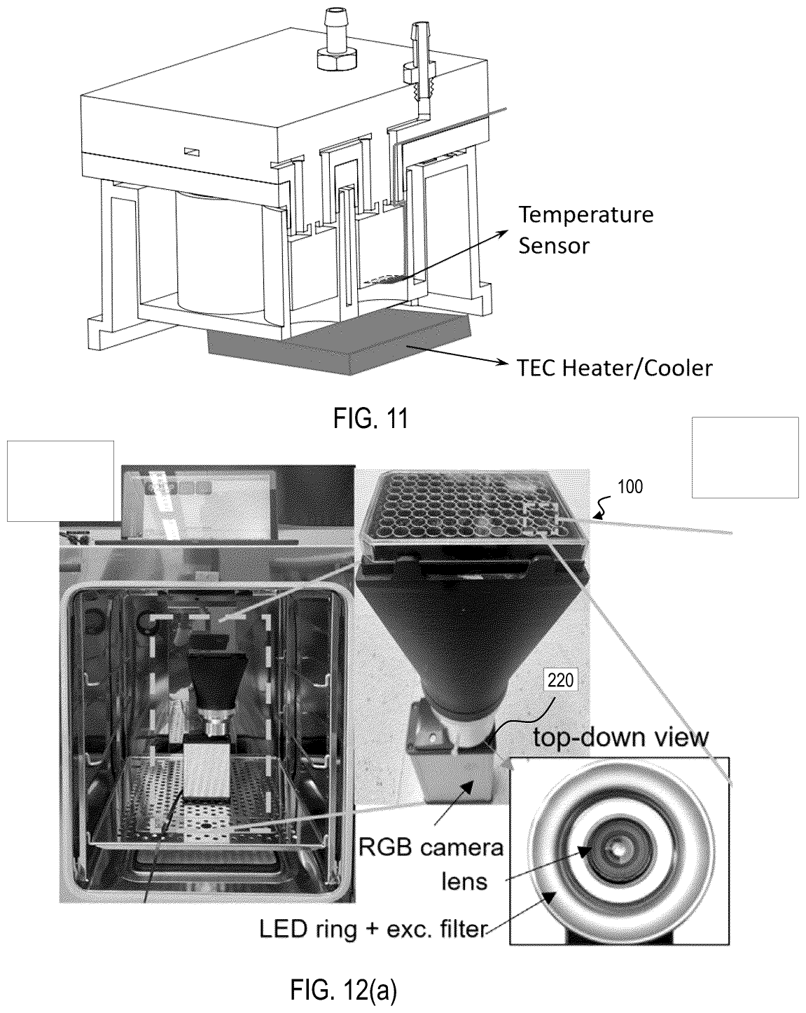

As shown, the system 200 provides a closed loop control system. That is, the closed loop automatically self-regulates (for example) the temperature, oxygen concentration and flow rate. The system has sensors (flow rate, oxygen, temperature, etc.) and feedback control of pump, oxygen input and heater/cooler (FIG. 11) provided by computer algorithm or hardware. In some embodiments, the media from the outlet connector 150b might contain wastes or other unwanted components. Accordingly, the media from the outlet connector 150b is not returned to the media reservoir 204, but instead is sent to a waste reservoir. In addition, as cells multiply or die they will use a different amount of oxygen, so the controller 210 can adjust the oxygen concentration accordingly, such as for example by adjusting how much oxygen is added to the media, especially if using only one reservoir 204, or by adjusting the flow rate. For two or more reservoirs, then the pressures change as the volumes change (e.g., input reservoir goes down and waste reservoir goes up), so the controller 210 can adjust to provide a constant flow rate.

The flow rate sensor 206 is in electronic (wired or wireless) communication with the controller 210. The pump is also in electronic (wired or wireless) communication with the controller 210, via the driver 212. The flow rate sensor 206 detects the flow rate of media at the cover assembly 100. That detected rate is sent to the controller 210, which can then adjust the flow rate by sending a flow rate control signal through the driver 212 to adjust the pump 202 to operate faster or slower. The controller 210 can also be in electronic (wired or wireless) communication with a temperature sensor (FIG. 11) and heater/cooler. The temperature sensor sends a detected temperature to the controller 210, which then sends a temperature control signal to the heater/cooler to adjust the temperate to be hotter or cooler. The heater/cooler can be located, for example, below the well plate.

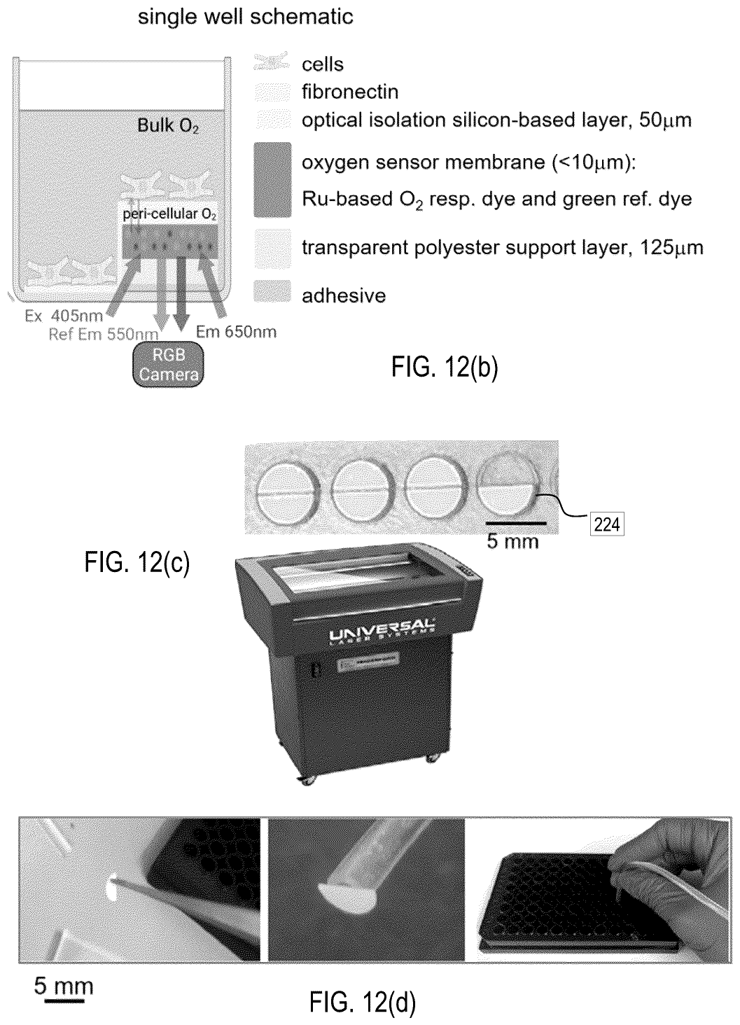

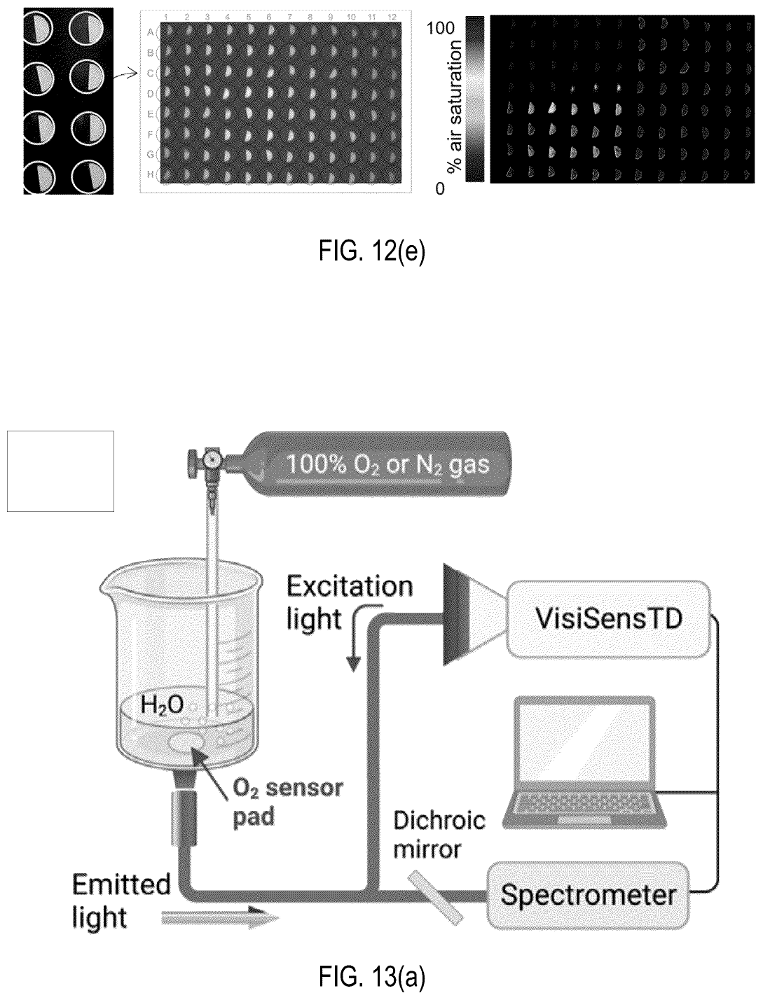

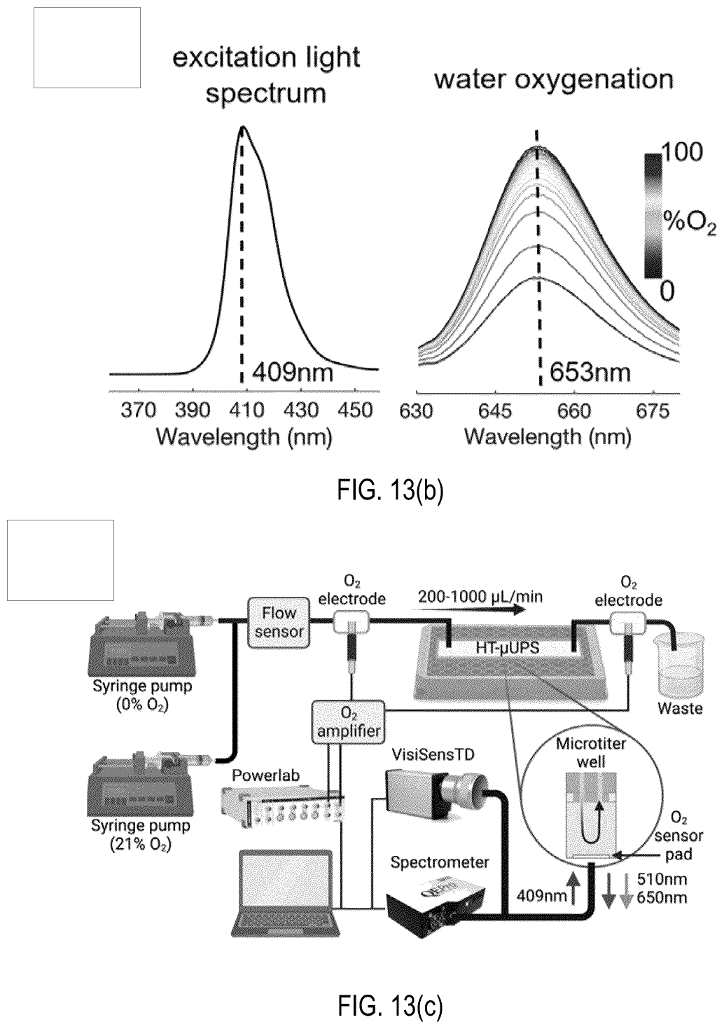

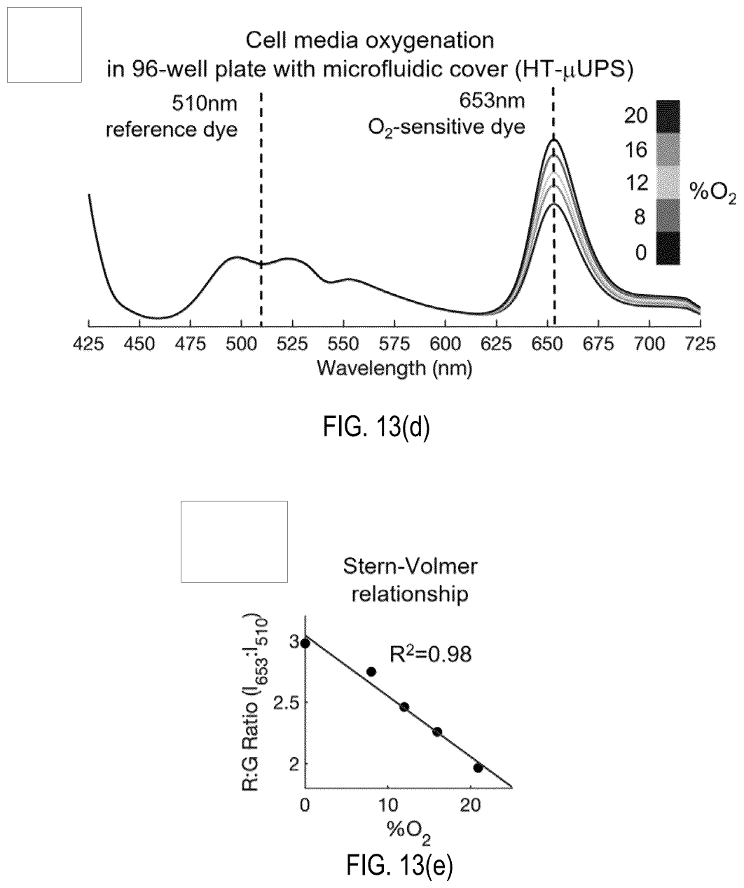

The controller 210 can also be in electronic (wired or wireless) communication with an oxygen sensor 224 (FIG. 12(c)) and light detector (e.g., camera, FIG. 12(a)) and gas supply (FIG. 13(a), 100% O₂ or N₂ gas). The oxygen sensors 224 are located in the wells, and emit red and green colors in response to the oxygen concentration level. That is, the sensors 224 are fluorescent and emit a fluorescence that is imaged by a camera 220. The fluorescent intensity ratio of two dyes in the sensor 224 indicates the oxygen concentration. In addition, the oxygen sensor can be configured to detect the concentration of other substances or gases, such as nitrogen, and the controller can adjust the nitrogen concentration based on the sensed nitrogen concentration. If the detected oxygen concentration is not the desired value, input oxygen concentration or media flow rate can be modified using a PID or PI feedback control mechanism until the detected oxygen concentration reaches the desired value.

The RGB (Red/Green/Blue) light detector, here a camera 220, detects or captures images of the sensors 224, and calculates the oxygen concentration of the media, as sensed by the oxygen sensor 224, and sends a detected oxygen concentration to the controller 210. The controller 210 then sends an oxygen control signal to the gas supply and/or the pump 202 to adjust the oxygen concentration to be greater or lower or by controlling the pump 202 to operate faster or slower (the faster the flow rate, the more oxygen that is supplied to the biofilm in the well, and the greater the oxygen concentration). Accordingly, the flow rate, temperature, and/or oxygen concentration are each separately detected and adjusted dynamically and in real time to keep the well at a desired temperature, flow rate, oxygen concentration level that best keeps bacteria of the biofilm alive.

In one embodiment, the flow rate sensor 206 model SLI-2000 (Sensirion) monitors flow rates. The piezoelectric pump 202 model mp6 (Bartel's Mikrotechnik) drives recirculating, unidirectional flow at 1 mL/min. The EVA OEM driver board 212 for this pump was powered by a standard USB type A port from a laptop 210. The driver was set to deliver a default amplitude of 270 Vₚₖ₋ₚₖ to the pump while the frequency of the pump was controlled using a Teensy 4.1 to generate a square wave varying from 30 Hz-150 Hz to maintain constant flow rate. The multi-well microtiter plate cover 101 has multiple components including plastic (e.g., polystyrene, COC, acrylic (PMMA), Polycarbonate, PDMS (see Tan, Kelly, Keegan, Philip, Rogers, Miles, Lu, Mingjian, Gosset, James R., Charest, Joe, Bale, Shyam Sundhar, A high-throughput microfluidic microphysiological system (PREDICT-96) to recapitulate hepatocyte function in dynamic, recirculating flow conditions, Lab on a Chip (19)9:1556-1566, 2019, which is hereby incorporated by reference) etc.) microfluidic channels, perfusion plugs, a elastomer (PDMS, PU, Fluoroelastomer) gasket, and connectors (e.g., barb-to-thread) to external tubing and reservoirs. The system fits within a standard laboratory incubator (FIGS. 1(c), 12(a)) and biosafety cabinet. The system is also portable for point-of-care applications such as on-site biospecimen preservation, on-demand biomanufacturing etc.

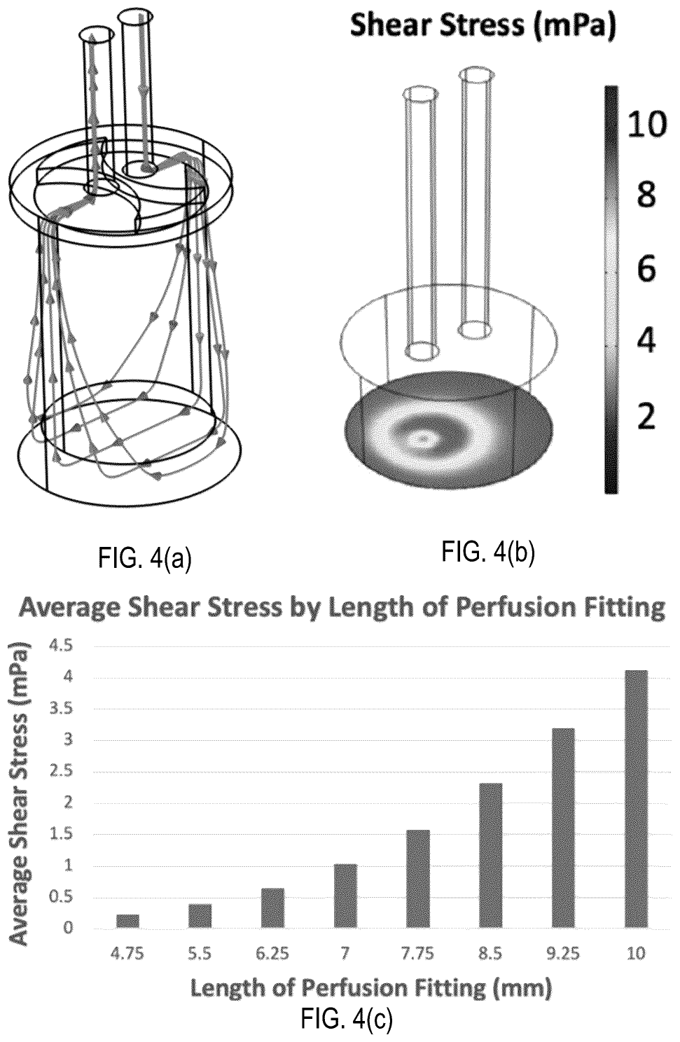

One or more connectors 150 (for example barbed connectors) are provided at the top layer 130 (or the top surface of the main layer 120 where no top layer is utilized). The connectors 150 connect directly or indirectly to the intake and exit openings. A tube can connect the connectors 150 to a pump or a reservoir. For example, an intake tube can be connected to the connector 150 at the intake channel 121. As shown by the arrows in FIGS. 1(a), 1(b), the inlet tube can introduce culture media, such as from a reservoir or tank, into the inlet channel 121. The media then flows down through the top layer 130 and main layer 120, through the perfusion plug 128, and exits at the exit opening of the inlet channel 121. The media 7 travels along the bottom portion of the well 112, where it contacts biofilm 5. The media 7 can flow in the space between the bottom of the well and the distal end of the perfusion plug 128. The media gets sucked up through the outlet channel 123. The media 7 travels up through the perfusion plug 128, the main layer 120, and the top layer 130, and exits through the connector 150 at the exit opening of the outlet channel 123. An outlet tube can connect a pump to the connector 150 at the exit opening of the outlet channel 123, so that the pump creates the media 7 flow. As shown in FIG. 4, the media spreads out to provide complete coverage of the biofilm 5 as it travels from the inlet channel exit to the outlet channel intake. In one embodiment, the connectors 150 are 3-56 brass thread-to-barb connectors from McMaster-Carr.

It is noted that not every well needs to be associated with a connector. Rather, a single inlet and outlet connector 150 can be associated with multiple wells 112. This is shown, for example, in FIG. 1(a), where the outlet channel 123 from a first well becomes the inlet channel 121 of an immediately adjacent well, and the outlet channel 123 of that adjacent well is connected to an outlet connector 150.

Still further, FIG. 1(b) shows the top layer 130 and the main layer 120 (including the perfusion plug 128), are formed as a solid cylindrical member, and the channels 121, 123 are formed therein, such as by drilling or using the top layer 130. However, the top layer 130 and the main layer 120 can be hollow (including the perfusion plug 128 can be tubular), and the channels 121, 123 can be formed as pipes that carry the culture media 7 through an opening in the main layer 120.

FIGS. 2(a), 2(b) show another example embodiment of the system 100. Here, the perfusion plug 128 of the main layer 120 has a different architecture. In FIG. 1, the perfusion plug 128 forms a media flow that starts and ends at the bottom center of the well 112. In FIG. 2, the perfusion plug 128 is configured to create a media flow, as shown by the arrows in FIG. 2(a), that starts at the upper portion of the well 112 at the sides of the well 112, travels down along the sides of the well 112 to the bottom portion of the well 112 and along the bottom of the well 112. This flow provides a more complete and uniform laminar flow and uniform shear stress of the media 7 along the bottom of the well. Accordingly, a uniform shear stress is applied to all of the biofilm 5, as in FIG. 4(a), compared to a non-uniform shear stress shown in FIG. 4(b). The main layer 120 has a support portion 124, as in FIG. 1. Here, however, the perfusion plug 128 has an upper portion formed as an extended neck 126, and a lower portion formed as the perfusion plug 128. A narrowed tab portion 127 connects the upper portion 126 and the lower portion perfusion plug 128. The upper portion 126 extends downward beyond the open top of the well 112 and into the well 112 interior. As further shown, the upper portion 126 has a distal end that extends at least as far as the distal end of the gasket 140, and here the distal end of the upper portion 126 is about flush with the distal end of the gasket 140, though the upper portion 126 can extend further than the gasket 140 or the gasket 140 can extend further than the upper portion 126. The perfusion plug 128 is wider than the upper portion 126.

As best shown in FIG. 2(a), the configuration of the perfusion plug 128 creates an inlet flow channel 121, base flow channel 119, and outlet flow channel 123. The inlet flow channel 121 is formed by a first inlet flow channel section 121a, an optional second inlet flow channel section 121b, third inlet flow channel section 121c, and fourth inlet flow channel section 121d. The outlet flow channel 123 has a first outlet flow section 123a, second outlet flow channel section 123b, an optional third outlet flow channel section 123c, and fourth outlet flow channel section 123d.

The first inlet flow channel 121a and fourth outlet flow channel section 123d are each an elongated opening that extends vertically through the main layer 120, top layer 130, and upper extended neck portion 126. The first inlet flow section 121a and fourth outlet flow section 123d each extend from the top surface of the top layer 130 to the distal end of the upper neck portion 126. The second and third inlet flow channels 121b, 121c and the optional second and third outlet flow channels 123b, 123c extend horizontally from the side of the well 112 to the narrowed tab 127, and orthogonal to the first inlet flow channel 121a and fourth outlet flow channel 123d, respectively.

The second inlet flow channel 121b and the third outlet flow channel 123c further extend from the narrowed tab 127 to the first inlet flow channel 121a and fourth outlet flow channel 123d, respectively, and between a portion of the top surface of the lower base portion 125 and a portion of the distal end of the upper neck portion 126. The third inlet flow channel 121c and the second outlet flow channel 123b extend from the side of the well 112 to the first inlet flow channel 121a and fourth outlet flow channel 123d, respectively. The fourth inlet flow channel 121d and first outlet flow channel 123a each extend vertically from the top surface of the lower base portion 125 to the bottom surface of the lower base portion 125, between the side wall of the well 112 and the side of the lower base portion 125, and orthogonal to the third inlet flow channel 121c and second outlet flow channel 123b, respectively.

The first inlet flow portion 121a and fourth outlet flow portion 123d are in flow communication with the second and third inlet flow portions 121b, 121c and the second and third outlet flow portions 123b, 123c, respectively. The third inlet flow portion 121c and second outlet flow portion 123b are in flow communication with the second and fourth inlet flow portions 121b, 121d, and the first and third outlet flow portions 123a, 123c, respectively. And the fourth inlet flow channel 121d and first outlet flow channel 123a are in flow communication with the bottom flow channel 119. However, in certain embodiments the second and third inlet flow channels 121b and 123c need not be provided, as shown for example in FIG. 2(c), which shows the system 100 with the channels 121b, 123c removed (filled).

### Media Flow

Referring to FIGS. 2(a), 4(a), 4(b), in operation, the media 7 flow is shown by the arrows. The media 7 enters the first inlet flow channel 121a (for example, from an inlet connector 150 or from a bridge or connecting channel 152 (FIG. 3(d)) from the outlet channel of another well 112) and travels downward to the optional second and third inlet flow channels 121b, 121c. Some media 7 may enter the second inlet flow channel 121b, as shown. The media 7 continues to travel in the third inlet flow channel 121c between the top surface of the lower base portion 12 and the bottom surface of the upper neck portion 126 (and the gasket 140), and then downward in the fourth inlet flow channel 121d into the entrance of the bottom flow channel 119. In some embodiments, the narrowed tab 127 need not be provided, and the upper neck portion 126 can be directly connected to the lower base portion 125, which eliminates the second inlet flow channel 121b and third outlet flow channel 123c. The media 7 travels along the bottom flow channel 119, where it interacts with the biofilm 5.

At the exit end of the bottom flow channel 119, the media 7 then enters the first outlet flow channel 123a, and travels upward to the second outlet flow channel 123b, and along the second outlet flow channel 123b, between the top surface of the lower base portion 125 and the bottom surface of the upper neck portion 126 (and the gasket 140). The media 7 then travels upward into the fourth outlet flow channel 123d. Some of the media 7 may also enter the third outlet flow channel 123c. The media 7 then exits the fourth let flow channel 123d to an outlet connector 150 (or to the inlet channel 121 of another well), and to a tube connected to a pump or other external device.

Thus, the perfusion plug 128 of FIGS. 2(a), 2(b), 3(c), is designed to have the media 7 flow directly along the sides of the well 112 from the top portion 114 of the well 112 (directly below the gasket 140) to the bottom of the well 112. Then along the bottom portion 118 of the well 112 directly along the sides of the well 112 from the bottom of the well 112 to the top portion 114 of the well 112 (directly below the gasket 140). That configuration provides a uniform shear stress distribution due to the media 7 along the biofilm 5 at the bottom of the well 112.

A specific example implementation of a system is discussed further below, without intending to limit the scope of the disclosure.

### Fabrication

The hard cover 101 can be designed in standard 3D CAD design software such as AutoCAD and Fusion 360, and the G-code for milling can be generated using Fusion 360. After cutting Two pieces of desired sized PMMA using a laser cutter Versalaser VLS 2.3 (Universal Laser Systems), a CNC machine (MDA V8-TC8) was used to drill all holes and mill all channels with spindle speed 18,000 RPM, feed rate 1000 mm/min, and depth of cut 0.5 mm. After milling, two pieces were thermal bonded using a heat press (Rosineer Grip Twist) at 130° C. for 3 hours, and the perfusion plugs were milled on the bottom side of the bounded piece using the CNC machine. After milling, threading was tapped and connectors were assembled, and the finished hard cover was cleaned using an ultrasonic cleaner (Branson 3800), pure water, and compressed air.

The soft gasket 140 was made by soft lithography from PMMA mold designed in Fusion 360 and fabricated using a CNC machine. After the mold was made, a thorough mixed PDMS mixture of Sylgard 184 and Dragon Skin with a 1:2 ratio was used to pour onto the mold and placed in a vacuum chamber to eliminate bubbles for an hour and then in an 80° C. convection oven overnight to cure. The Cured soft gasket is carefully peeled off the mold and cleaned using an ultrasonic cleaner, water, and compressed air. (See Supplementary material for relevant CAD drawings of each component).

### Computational Fluid Dynamics (CFD) Simulation Setup and Result

Biofilms found on implanted medical devices such as urinary catheters are commonly subjected to environmental shear stresses (Stickler 2008). Different shear stress conditions can alter Biofilm formation and growth rates (Thomen 2017). To estimate the shear stress applied to the biofilm in the bioreactor system, computational fluid dynamics simulations of streamline, linear velocity, and shear stress were performed.

CFD simulations were performed using COMSOL Multiphysics 5.5 software, and the simulation results were analyzed using MATLAB. The 3D model used in the CFD simulation was designed based on the dimension of the biofilm reactor and the standard 96-well microplate. To study the relationship between the depth of the perfusion plug 128 on the microfluidic cover and the shear stress at the bottom of the well plate, we simulated the shear stress at the bottom of the wells for the different perfusion plug depths ranging from 4 mm to 10 mm.

The simulation is based on Reynolds-averaged Navier-Stokes equations (Turbulent flow K-ω interface). The boundary conditions for inlet and outlet was set as fully developed flow with volume flowrate V_{O}=1 mL/min, and that for other boundaries was set as No-slip. The mesh had an extra-fine element size with minimum element quality <0.002. The simulations were done on an Intel® 64 bit CPU (Intel® Core™ i9-9900KF CPU @ 3.60 GHz, family 6, model 158, stepping 12, 8 cores with 64 G RAM) running a Windows® 10 operating system. The simulation took ten hours and fifty-three minutes.

Referring to FIGS. 4(a), 4(d), the CFD simulation result for streamline showed that the biofilm reactor perfusion cover can provide uniform laminar flow to each well, enabling efficient media circulation for biofilm growth in the system. The results also showed the CFD simulation result for the linear velocity distribution in a well; the result shows the absolute linear velocity experienced by biofilm in the center of the well varies minimally to promote biofilm generation. The average shear stress experienced on the bottom of each well with different lengths of perfusion plugs on top. The shear stress is higher when the perfusion plug is longer and closer to the bottom of the well. Accordingly, the dimensions (e.g., length, diameter/width) of the perfusion plug, as well as the media and flow rate can be varied depending on the application, i.e., the well size, number of wells, and target material being tested.

### Cell Culture

Lyophilized E. coli (ATCC 25922) cells were purchased from ATCC and resuspended in LB broth (Sigma 13522) before streaking onto LB agar plates (Carolina Biological 216600). After 24 hours of incubation at 37 C, plated colonies were inoculated by sterile pipet tip in 10 mL of sterile LB broth and incubated for an additional 24 hours. These cultures were then diluted to an OD600 of 0.2, and 200 μL of diluted culture was transferred to each corresponding well in a 96 well plate. This plate was then incubated at 37 C for 1 hour and examined under brightfield microscopy before attaching the cover.

### Plate Reader and Microscope Setup

All plate reading experiments were conducted using a SpectraMax M2 plate reader (Molecular Devices) and the Softmax Pro 6.2 software. This platform was used for both biomass and viability testing. An Olympus IX71 microscope was used along with 10× objective and X-Cite Series 120 (Lumen Dynamics) fluorescent source was used to collect images to assess viability. FITC and Rhodamine filters (Chroma) were used to view SYTO9 and Propidium Iodine dyes, respectively.

### Culture Preparation for Evaluation

Following cell culture, all bacterial suspension was removed from the 96 well plate 110 and replaced with fresh LB broth. All system components were assembled and primed with LB broth. The system was controlled outside of the incubator (FIGS. 1(c), 12(a)) through a laptop computer 210, and flow rate was monitored and recorded using the USB RS485 Sensor Viewer application. Following 24 hours of culture, the system was removed from the incubator and placed in a secondary container before removing the cover for optical evaluation. All media was removed from each well in the 96 well plate and wells were washed by pipetting three times with deionized water. Optical evaluations included brightfield microscopy, fluorescence-based viability testing, and absorbance-based biomass testing. Tubing and reservoir components were subsequently discarded while the gasket and pump components were first washed in 70% ethanol before steam autoclaving at 121 C for 30 minutes. The acrylic cover and flow rate sensor were washed with 70% ethanol for fifteen minutes before flushing with deionized water and allowing to dry.

### Cell Biomass and Viability Test

Biomass evaluation was performed using a modified protocol from O'toole (2011). Wells were stained with 50 μL of 0.1% v/v Crystal Violet for 15 minutes then washed by pipetting three times with deionized water. After drying, brightfield images were taken of the well plate and the crystal violet dye was solubilized with 200 μL of a 30% v/v acetic acid solution. The absorbance of each solubilized well was measured using a plate reader set to 550 nm.

The viability test was performed using an L7012 LIVE/DEAD BacLight kit from Thermo Fisher. The protocol was followed as developed by Molecular Probes (Invitrogen 2004). Prior to adding fluorescent dyes, a small subset of wells was selected for treatment with 200 μL of 70% ethanol for 45 minutes. The dyes SYTO9 and Propidium Iodine were mixed in a 1:1 ratio and diluted in deionized water. 100 μL of dye was added to each well and allowed to incubate at room temperature in the dark for 30 minutes. Each well was then emptied and rinsed with deionized water and allowed to dry. Fluorescence measurements were first taken from an epi-fluorescent microscope and all images were processed in MATLAB 2019b. Measurements were then taken from a plate reader set for excitation/emission spectra of 485 nm/530 nm and 485 nm/630 nm for SYTO9 and PI, respectively. Viability was determined by comparing the ratio of emission from SYTO9 versus PI.

### Statistical Analysis

All statistical calculations were done in MATLAB 2019b using the built-in statistics toolbox. For both biomass and viability experiments, a one-way analysis of variance (ANOVA) was performed with a significance level α=0.05 followed by multiple comparison tests. These comparisons were made using the Tukey-Kramer post hoc test due to the unbalanced sample sizes between groups.

### Different Shear Conditions

A cover 101 with different length of well fittings was fabricated to deliver perfusion of media to sixteen wells in series. The results of shear stress simulations are shown in FIGS. 4(a)-4(c). The configuration of the cover 101 provides different shear conditions. Additionally, two wells containing pure media containing no bacteria as well as two wells cultured with bacteria but under no flow were plated on a 96 well plate. A flow rate of 1 mL/min was chosen to deliver average shear stress values as determined from a previous culture in microfluidic channels (Thomen 2017). Furthermore, these values are consistent with recommended shear conditions for urinary catheter infection models (Ramstedt 2019). The peak system flow rate achievable was 3.7 mL/min which was not used for bacterial culture presented here but could enable future studies at high shear. The biomass results following 24-hour perfusion suggested an increasing trend of biofilm formation with increasing shear stress up until the well with highest shear stress. From these results, a well-fitting height of 9.25 mm was chosen for the full-scale 96 well cover design.

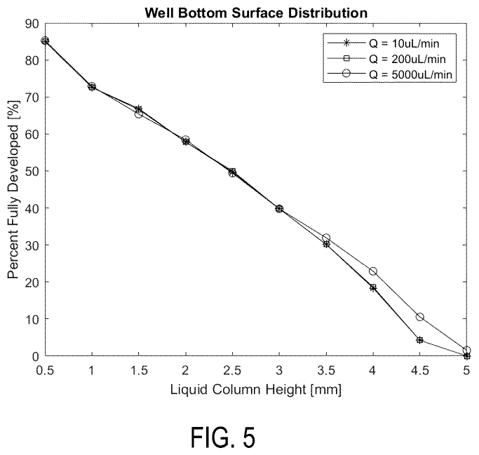

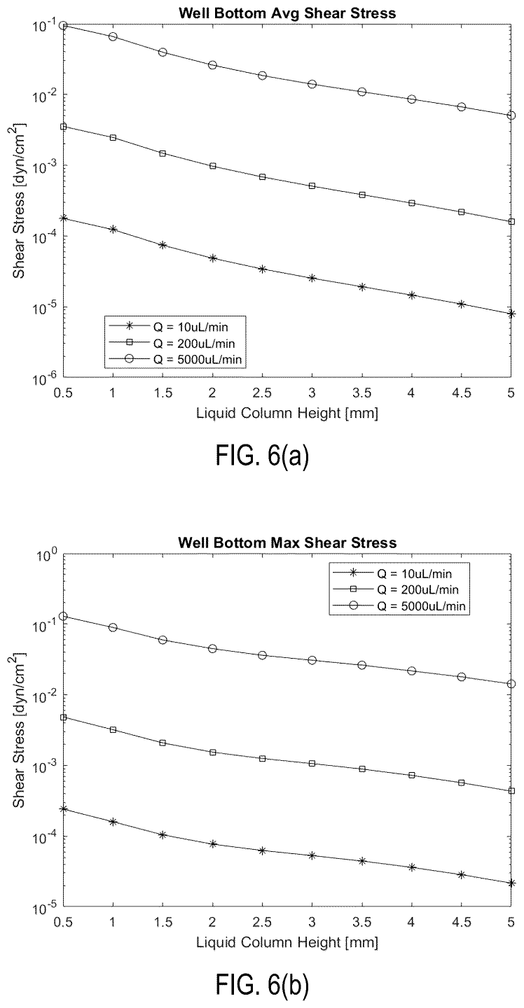

By increasing the length of the well-fitting (i.e., decreasing the liquid column height), the percentage of fully-developed laminar flow can be increased, which will result in more uniform flow velocity and shear stress at the well bottom, as shown in FIGS. 2(a), 5. In addition, as shown in FIG. 6, the desired shear stress magnitude (e.g., average shear stress across the well bottom (FIG. 6(a)), or maximum shear stress at the center of the well bottom (FIG. 6(b)) can be achieved by changing the liquid column height and flow rate. In one embodiment, the height of the media flow at the bottom portion 118 (between the well bottom and the distal end of the perfusion plug), is 200 micron to 10-millimeter height, such that the perfusion plug blocks up to 98% of the well volume 112; though in some embodiments up to 80%, in some embodiments up to 90%, in some embodiments up to 95%, and in other embodiments up to 98%.

### Biofilm Biomass

Biomass testing with crystal violet staining in microtiter plates has gained popularity due to its throughput and convenience. The system 100 is compatible with this staining technique. A 24-hour biomass assay was performed with a full 96 well cover. Forty-eight wells were utilized for this test with eighteen being perfused, eighteen covered but non-perfused used as a control, and twelve left uncovered and cultured under static conditions. Two of the uncovered wells contained pure media and were used as a negative control. The non-perfused wells were used as a control to observe any effects of covering the wells. The uncovered wells were used as a comparison with a more standard microtiter biofilm assay (O'toole 2011). A statistical significance at α=0.05 was observed with p<0.001 between both Perfused and Non-perfused and Perfused and Uncovered groups. No statistical significance was observed between non-perfused and covered groups. These results demonstrate the throughput of the cover and potential of the system of the system to interface with a standard well plate, plate reader, laboratory incubator and microscope.

### Biofilm Viability

Biofilm viability was determined optically with fluorescent based detection for groups of perfused and non-perfused wells. Some cells (i.e., bacteria in the biofilm) are dead, some are alive] Two fluorescent dyes SYTO 9 (green) and Propidium Iodine (red) are used to label live and dead bacterium cells respectively. Cell viability was determined by comparing the ratio of fluorescent emission from SYTO9 versus Propidium Iodine. Images taken from microscopy (FIG. 12(a)) showed marked differences between perfused and non-perfused groups as well as between perfused wells and those treated with ethanol. Ethanol treated samples served as the dead bacteria control. Perfused wells were compared with control wells (covered but non-perfused wells) to examine any effects of perfusion. Viability testing was performed in addition to biomass staining as crystal violet does not indicate the sample viability. Perfused wells showed the highest ratios indicating relatively higher viability compared to other groups. Ultimately, this result demonstrates the system's capability to integrate with standard fluorescent assays for microtiter plates. Values obtained from a fluorescent plate reader agreed with these images showing a statistical difference between perfused and all other group (p<0.001) indicating perfused wells have more viable bacterium cells. No statistical difference was observed between non-perfused wells treated and untreated with ethanol, which indicates that the cells in nonperfused wells are mostly dead. Thus, by having a uniform flow, more oxygen is supplied to the cells, so that the perfused wells are better and result in more viable cells.

### Conclusion

In summary, a novel perfusion biofilm reactor is provided that is compatible with standard 96 well plate and high throughput in situ optical evaluation. That is, a 96-well (or greater, e.g., 384, 1536) format provides high throughput because you can do 96 tests simultaneously. And a standard well plate (i.e., a planar plate with one or more wells having set dimensions (length/depth, width/diameter)) can be utilized to operate with the current system, and the well plate need not be modified or customized, and the cover 101 need not be removed (so that the growth environment can be maintained by avoiding exchange of gases that would otherwise occur when the cover is removed). While the focus of this work was a 24-hour endpoint assay, the system allows for chronic and real-time microscopic evaluation, provided the inclusion of an on-stage incubator. Among the advantages for this system are automatic priming and closed-loop flow rate control. Automated priming of the system simplifies and improves the consistency of repeated experiments. Our previous methods for manual priming involved either gravity or syringe-based fluid delivery. However, when performed manually, these methods can be tedious and prone to error due to changing liquid height levels and inconsistent pressure applied to the syringe (data not shown). The closed-loop feedback control of the pump allows for more consistent system priming without the need for manual effort.

Furthermore, Azerado et al. have reported clogging as an issue for microfluidic bacterial culture (2017), and prior to the implementation of closed-loop flow rate control, we observed difficulty in maintaining the desired flow rate for the entire culture period. We attribute this difficulty to the effect of biofilm growth in microfluidic channels and tubing leading to increasing resistance over time.

A summary of our device's performance as compared to other published and commercially available alternatives is shown in table 1. Other commercially available 96-well format bioreactor systems such as Predict96 and BioFlux require significant modification of the well-plate to achieve perfusion (Fluxion 2008; Draper 2018). In contrast, our system involves no modification to the 96 well plate to achieve high throughput, customizable flow conditions. In its current configuration, wells are serially connected in a single row, but each row may hold a different condition, though it is possible to chain rows together with tubing for greater sample size as demonstrated in this work. These different conditions may come in the form of different antibiotics or media formulations, to give two examples. However, to achieve recirculation of different conditions without cross-contamination, some modification of the fluid scheme will be required through the addition of pumps or valves.

Several improvements to this technology may be considered for future study. The material choice for the cover of PMMA limits sterilization methods required for reuse. While methods of ethylene oxide and hydrogen peroxide have been demonstrated effective without harming the material (Yavuz 2017), further testing is required to ensure repeatability. Furthermore, the non-uniform shear condition across the bottom can be addressed by altering the geometry and flow path of the well-fitting. We plan to study this further in future work by first modeling alternate geometries that modify the flow path within the chamber to achieve a more fully developed flow. These geometries will be simulated in COMSOL at different plug heights and flow rates to determine an optimal shear stress uniformity. Additionally, longer-term studies should be performed when investigating the roles of oxygen delivery, nutrient delivery, and shear stress on biofilm biomass. Lastly, the system would benefit from a precise gas control as the availability of oxygen has been shown to affect bacterial biofilm growth (Cui 2004; Adler 2012). To adapt to situations requiring oxygen control, we would propose two strategies. The first would be to add active oxygenation to the reservoir to prevent depletion while using closed loop recirculating flow. The second strategy involves modification of the perfusion plug length to alter oxygen delivery time. We may further optimize this time while considering the bottom shear stress through simulation modeling.

Aside from its use as a biofilm reactor, this system 100 has applications with targets other than biofilm, such as for example, cells, in antibiotic susceptibility testing (Macià 2014; Blanco-Cabra 2021), extracellular polymeric substance biogenesis (Barnhart 2006; Serra 2013; Kan 2019), and pharmacokinetic and pharmacodynamic studies (Hengzhuang 2012; Cao 2015), as well as drug development, fundamental biological research, cell culture, tissue engineering, antibiotic susceptibility testing. Each of these areas may benefit from the availability of automated perfusion with well controlled flow and optical evaluation in situ.

It is noted that the system 100 is shown and described to apply a uniform fluid shear stress to a biofilm 5. It will be apparent, however, that the system 100 need not be used for a biofilm in a well, but rather can be applied to any suitable material or components, such as for example, cells, tissue, organoids, spheroids, either at the well bottom, elsewhere in the well, or in a different container.

One important application of the system 100 is to achieve a high-throughput microfluidic perfusion biofilm reactor (HT-uPBR) that is compatible with a standard 96-well microtiter plate and in situ optical evaluation techniques. The reactor includes multiple subsystems including a fluid reservoir, piezoelectric micropump, flow rate sensor, 96-well plate cover, and electronic controls. The 96 well plate cover has multiple components including polymethyl methacrylate (PMMA) microfluidic channels, PMMA perfusion plugs, a polydimethylsiloxane (PDMS) gasket, and stainless steel barb-to-thread connectors. The system fits within a standard laboratory incubator and biosafety cabinet.

The device has been characterized using computational fluid dynamics (CFD) modeling in COMSOL multiphysics software. A prototype of the system was fabricated and tested by culturing E coli. bacterial biofilms in a standard 96-well microtiter plate. The perfusion system was then connected to the plate and allowed to perfuse 24 hours before determining biofilm biomass and viability. Results of the culture showed significantly greater biofilm growth on the perfused well bottoms. Other features include modification of the fluid control to prevent cross contamination between wells, modification of the cover geometry to increase the uniformity of fluid shear stress to the well bottom, and the addition of precise gas control.

FIGS. 3(a)-(d) show an example uniform shear cover on a 96-well plate. Here, a serial flow path is used, whereby media 8 flows from one of the inlet connectors 150a to a respective one of the outlet connectors 150b. FIG. 3(a) is an assembled perfusion cover on a standard 96-well plate. FIG. 3(b) is an exploded view of a perfusion cover and a PDMS gasket on a standard 96-well plate. FIG. 3(d) is an exploded cut-through view of a perfusion cover on a microtiter plate, with only two wells in the plate shown. And FIG. 3(e) is a side view of a perfusion cover on a standard 96-well plate.

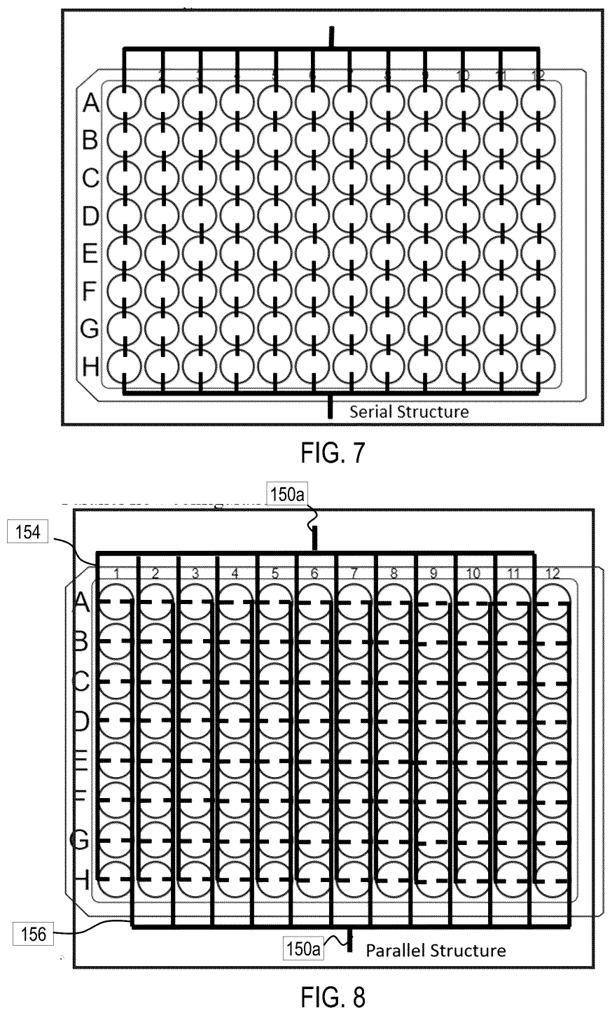

FIGS. 3(d), 7, 8, 9 show various design configurations for the well plate 110. FIG. 3(a), 3(b), 3(d) show a separate input and output connector 150a, 150b for each row of wells 112; whereas FIGS. 7, 8, 9 show a single input connector 150a and a single output connector 15b for all rows/columns of wells. FIGS. 3(a), 3(b), 3(d), 7, 9 show serial flow of media 7 from the inlet connector(s) 150a to the outlet connector(s) 150b. More specifically, the media 7 travels from each of the inlet connector(s) 150a to a first well 112. The media 7 exits the outlet channel 123 of the first well 112, enters the bridge channel 152, then exits the bridge channel 152 and enters the inlet channel 121 of a second well 112 in the same row (FIG. 3(d)) or column (FIG. 7) as the first well 112. At the last well 112 in that row or column, the media 7 travels from the outlet channel 123 to the outlet connector 150b.

FIG. 8 shows a parallel structure of wells 112. Here, the media travels from an input connector 150a to an inlet supply channel 154, through a plurality of wells 112, to an outlet supply flow channel 156. The same inlet supply channel 154 can feed one or more wells 112, and the same outlet supply flow channel 156 can receive outlet from one or more wells 112 and provide a combined outlet to the outlet connector 150b.

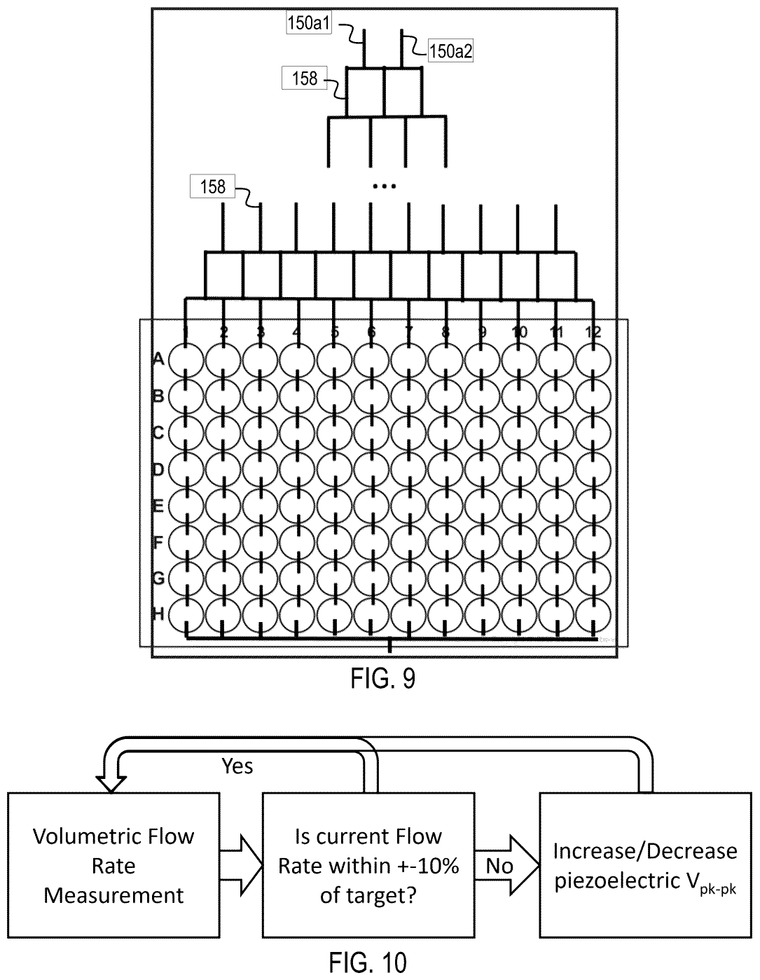

FIG. 9 shows another example embodiment, whereby perfusion is integrated with a concentration gradient generator (e.g., using a serial configuration as an example). Media enters at the top and is split into multiple dividing channels and can travel through multiple tiers of channels to form a flow channel tree, as shown. Each successive level creates a different concentration of media. For example, a first inlet connector 150al can receive 100% oxygen from a first media source (gas supply), and a second inlet connector 150a2 can receive media with 0% oxygen from a second media source. The first and second inlet connectors 150a1, 150a2 both connect to a first tier of three branch channels 158, which in turn connect to a second tier of four branch channels 158, etc. In this embodiment, each tier has one additional branch channel 158 than the last tier, so each tier creates branches with different oxygen concentrations.

Thus, when those media combine at the second level, the left channel has a media with 100% oxygen, the media at the middle channel has 50% oxygen, and the right channel has a media with 0% oxygen. Each successive level has a preliminary or branch channel 158 with additional percentages of oxygen. Thus, the media travelling through the respective wells can test the biofilm 5 for different properties, such as a different oxygen concentration or gradient of media. It is noted that the oxygen does not diffuse quickly. As a result, for example, at level 2, the laminar flow will not mix the two input liquids thoroughly so the left channel will end up still ˜100%, middle channel ˜50%, right channel ˜0%. It is noted that other suitable configurations can be provided, such as for example, certain channels can extend directly to the cover apparatus 100.

FIG. 10 shows closed-loop volumetric flow control. Volumetric flow control system includes hardware (such as MCU, FPGA, computer, pumps, flow rate sensors, valves such as solenoid flow through or pinch vales, tubing, reservoirs etc.) and software including firmware for closed-loop flow rate control and GUI (e.g., Python based) for sensor data display, logging, and manual control. A flow rate sensor, for example, is used to measure the volume flow rate inside the system and the sensor data is sent to a microcontroller through an USB or I2C or SPI interface, if the flow rate is different from the desired set point, the microcontroller can send command to the micropump to change its output flow rate or pressure. A proportional-integral-derivative (PID) or PI or simple on/off control algorithm can be implemented in the microcontroller firmware to achieve a stable desired flow rate in the system.

Interfaces with firmware and electronics to control the micropump (e.g., a mp6 piezo micropump, or a small DC brushless motor pump, or a miniature syringe pump or a miniature peristaltic pump). For example, a piezo micropump (e.g., mp6) flow rate can be controlled with inputs of square wave frequency and peak-to-peak voltage. Different feedback control methods such as proportional-integral-derivative (PID) or PI or simple threshold-based controller can be used based on the applications. The GUI and control software accept as an input a running log of flow rate data, such as that outputted from the flowrate sensor (e.g., Sensirion Sensor Viewer software)

FIG. 11 shows closed-loop temperature control. A miniature thermocouple or thermistor or semiconductor IC temperature sensor can be attached (e.g., adhered or glued) to the cover 101 to measure the liquid temperature near the cells/tissue/organoids/spheroids, and a thermoelectric heater/cooler or a resistive heater below the microtiter plate can be used to control the in-well temperature at the desired value (e.g., 37° C.) via a PI or PID or threshold-based on/off feedback control algorithm. For example, a sensor wire can travel internally through, or externally along, the main layer 120 and perfusion plug 128, to the outside surface at the distal bottom end of the perfusion plug 128 to directly contact the media flow at the bottom portion 118 of the well 112. The temperature sensed by the sensor can control operation of a heater/cooler positioned below the well plate 110, for example. Other sensors can be provided, such as an oxygen sensor that is used to control the amount of oxygen in the media.

Pericellular oxygen sensing. Closed-loop peri-cellular oxygen control to generate normoxia, hypoxia and hyperoxia conditions near cells/tissues/organoids/spheroids/biofilms. Combined pericellular oxygen concentration sensing, close-loop flow control, input Oxygen level and cover design, the system can generate different oxygenation conditions near the cells/tissue slice/organoids/spheroids. For example, an oxygen bubbler can be used to control the oxygen concentration (from 0%-100%) in the input reservoir. A concentration gradient generator (FIG. 9) can be used to generate different peri-cellular Oxygen concentrations in different wells. In addition, by changing the cover insert depth and cover insert shape and flow path, different peri-cellular oxygen distribution can be further controlled.

### Pericellular Shear Stress Control (Uniformity and Magnitude)

An example uniform shear cover design is shown in FIGS. 2, 3 and 4. The cover insert has a multi-layered structure which diverts the input liquid to flow along the side wall of the well and then enter the bottom as a parallel-plate flow pattern, resulting in a nearly uniform shear distribution. The magnitudes of the shear stress can be controlled by changing either the cover insert depth or input flow rate.

Compatibility with standard microtiter plates (e.g., 96-well plates). Compatibility with standard laboratory microscopy and microplate based processing and analytical instruments. Other parameters in the wells such as pH, glucose concentration, drug concentration and their temporal profiles can also be controlled using similar closed-loop sensing+flow rate adjustment approaches. Other commercially available microtiter plate format bioreactor systems such as Predict96 (Draper) (https://pubs.rsc.org/en/content/articlelanding/2019/lc/c81c01262 h, https://www.draper.com/explore-solutions/predict96) and BioFlux (Fluxion) (see Fluxion, Growth of oral and environmental biofilms using the BioFlux System. (2008). https://www.il-biosystems.com/fileadmin/user_upload/products/Fluxion/BioFlux_200/BiofilmGrowth.pdf, which is hereby incorporated by reference) require significant modification of the well-plate to achieve perfusion. These modifications make the plates incompatible with common optical analysis techniques such as a plate reader. These modifications also increase the cost and size of the systems due to the need for specialized pneumatics and integrated imaging. In contrast, our system involves no modification to the microtiter plate to achieve high throughput, customizable flow conditions and flow is achieved through an electronically controlled miniature pneumatic system. Therefore, our system offers superior flexibility for analysis options. As opposed to static flow reactors, our system can provide continuous perfusion to provide physiologically relevant shear stress, oxygen and nutrients supply, and waste removal. Desired drug and signal molecule profiles can also be provided by programmable perfusion.

This system is novel in comparison to convention systems in three main aspects: (1) other microplate perfusion bioreactors doesn't provide uniform shear stress to cells/tissues/organoids/spheroids; (2) this system offer closed-loop peri-cellular Oxygen monitoring and control not available in any existing microplate perfusion bioreactors to our knowledge; (3) the materials and manufacturing methods of the perfusion cover in this system is different from that of prior perfusion covers that only use PDMS. The present perfusion cover can be made of many different plastics such as PMMA, COC, polystyrene, PDMS or the hybrid combination of them.

### Optical Oxygen Sensors and Peri-Cellular Oxygen Imaging

Referring to FIG. 12(a), the system 200 is of FIG. 1(c) can further be configured for oxygen concentration sensing. The system 200 includes a camera 220 and an oxygen sensor, which is located in the well 112 of the well plate 110 of the apparatus 100. The bottom of the well is transparent, so that the camera 220 can capture red/green from the oxygen sensor and use an algorithm to determine the concentration of the oxygen in the well. Peri-cellular oxygen was measured using emission ratiometry. An integrated system comprised of an LED light source and an RGB camera (VisiSensTD, PreSens) imaged changes in the luminescence of optical oxygen sensors placed at the bottom of each well of a 96-well glass-bottom plate (FIG. 12(a)). The plate was placed on top of the VisiSensTD system for continuous monitoring in a cell culture incubator. The oxygen sensor membrane (FIG. 12(b)) incorporated an oxygen-responsive ruthenium dye and an oxygen-insensitive reference dye. Blue light from the LEDs positioned around the camera lens excited the two dyes. The camera imaged the entire plate to acquire oxygen-dependent changes in the luminescence ratio of the two dyes within the sensor located in each well of the plate.

The oxygen sensors can be, for example, semicircles that cover up to about one-half of the glass bottom of each well. They are laser-cut from a larger sheet (PreSens SF-RPSu4) that includes an oxygen sensitive layer, a polyester support layer, and a white optical isolation layer, which was placed onto a sacrificial acrylic sheet with the adhesive facing up (FIG. 1(c)). Semicircular sensors were then laser-cut using a 30 W CO2 laser (Universal Laser Systems VLS 2.3) by placing the acrylic layer on the laser cutter bed and focusing the laser on the top of the acrylic layer. The laser cutting path was drawn in AutoCAD 2022 to cut semicircles with a radius of 2.5 mm using 15% maximum laser power and 10% maximum speed.

It is noted, however, that the oxygen sensors can be any suitable size and shape, and need not be semicircles. In some embodiments, the oxygen sensors can be transparent, such as for example the two dyes can be embedded in a transparent material such as PDMS, so that no portion of the transparent well bottom is blocked. Thus, the oxygen sensor does not interfere with the camera or microscopic imaging (including fluorescence). In other embodiments, the oxygen sensors can be semi-transparent. In still other embodiments, the oxygen sensors can be opaque but sufficiently small so as not to interfere with the camera or microscopic imaging, such as for example, blocking about half of the transparent well bottom, though it is desirable to have the oxygen sensor block a minimal amount of the transparent well bottom.

### Oxygen Sensor Attachment and Plate Sterilization

Oxygen sensors are attached to the bottom of the wells of 96-well plates using sterile procedures inside a laminar flow hood. Sensors were lifted with tweezers to expose the white optical blocking layer while placing a suction tube (ID<2 mm) against this layer to hold the sensor while lowering it into a well (FIG. 12(d)). The suction in the tube was released once the adhesive layer attached to the glass. The process was repeated to place sensors in each well (FIG. 12(e)). Before using the plate for a cell culture experiment, the wells were sterilized with 70% ethanol inside the sterile laminar flow fume hood. After the ethanol evaporated, each well was washed three times with 1×PBS, before coating with fibronectin. After an experiment, raw images acquired by the VisiSensTD system were processed using a two-point calibration to convert the RGB values for pixels that imaged each sensor to a percentage corresponding to the peri-cellular oxygen level. An example raw luminescence image showing the optical sensors in a 96-well plate and the processed oxygen reading is illustrated in FIG. 12(e).

### Oxygen Sensor Spectral Characterization

Spectra for the full range of aqueous oxygen concentration were first measured by bubbling a beaker of water with 100% O₂ gas followed by bubbling with 100% N₂ gas (FIG. 13(a). A sensor attached to the bottom of the beaker was illuminated with excitation light from the VisiSenseTD system and the spectrum of emitted light was acquired once every second using a spectrometer (Ocean Optics QE-Pro, FIG. 13(b). FIGS. 13(a), (c) are used to calibrate the oxygen sensors.

Spectra for oxygen concentrations that are typical for cell cultures were acquired using a similar approach and our high throughput microfluidics-based uninterrupted perfusion system (HT-μ UPS) cover for a 96-well plate (FIG. 13(c)). Nitrogen-bubbled cell culture media (which has 0% O₂) and media equilibrated in room air (21% O₂) were loaded into two separate syringes that were each placed in one of two syringe pumps (New Era 1600X2). Media from each syringe flowed through a sensor (Sensirion SLI-2000) that measured the flow rate as media moved through our HT-μUPS cover to perfuse the wells of the plate (FIG. 13(c)). Flow-through Clark electrodes (Microelectrodes Inc. MI-730) incorporated into the tubing before and after the plate measured the media oxygen concentration at those positions. The system flow rate was maintained at 200 mL/min while the flow rates of the two syringe pumps were varied to achieve mixtures of 0, 8, 12, 16, and 20 percent O₂, which were confirmed by the Clark electrodes. The same spectrometer and fiber optic cable setup as in FIG. 13(a) was used to acquire the luminescence spectrum of a sensor within one well once every second.

Spectra collected during each characterization were plotted together to visualize changes in spectral bands corresponding to the oxygen-insensitive reference dye (centered at 510 nm, “green”) and the oxygen-responsive ruthenium dye (centered at 653 nm, FIG. 13(d). Values at the peak wavelengths for red and green luminescence (FIG. 13(d), dashed lines) were used to construct the inverse Stern-Volmer relation in FIG. 13(e). For this relationship, the ratio of red to green emission was plotted against each oxygen concentration measured by the Clark electrode placed before the plate. A linear fit of ratio versus oxygen concentration was used due to the small oxygen concentration range of 0-20% that corresponds to typical cell culture conditions.

### Oxygen-Impermeant HT-uUPS Cover and Temporal Characterization

The responsiveness of the oxygen sensing system to changes in media oxygen concentration and flow rate was characterized using 96-well plates and a new oxygen-impermeant version of the HT-μUPS cover described in our previous work. This cover was assembled in two components, one soft PDMS sealing gasket and one acrylic perfusion base that was CNC-milled from cast acrylic (McMaster-Carr 8560K354) and thermally bonded using a heat press (Rosineer Grip Twist) at 130° C. for 3 hours. The sealing gasket was fabricated by pouring a mixture of Sylgard 184 (Dow) and Dragonskin 10 (Smooth-On) in a 1:2 ratio into an acrylic mold. Finally, an inlet and outlet were tapped (10-32 thread) for each well and fitted with stainless steel 10-32 barb-to-thread connectors (Pneumadyne).

The sensor response within the perfusion system HT-uUPS was validated. Clark electrodes were placed before and after the cover. An optical oxygen sensor was placed at the bottom of the leftmost well of the cover. The Clark electrodes and VisiSensTD system were calibrated with water at 100% O2 and 0% O₂. Water bubbled with 100% O₂ was loaded into a syringe pump and perfused through the cover. Flow rate was measured using a flow meter (Sensirion SLI-2000) and varied between 500 mL/min and 200 mL/min to determine the system's response to changes in flow.

### Two-Point Oxygen Calibration

A two-point calibration converted the ratio of red to green luminescence intensity imaged from the sensors into percentage of air saturation. The terms ‘Cal0’ and ‘Cal100’ were used in system characterization experiments for 0% and 100% air saturation, and oxygen saturation levels ranging from 0% to 18.6% were displayed in cell experiments (the equilibrium oxygen partial pressure in a humidified 37° C., 5% CO₂ incubator is 18.6%).

Two types of culture media, CDI iCell Cardiomyocytes² maintenance medium (Fujifilm CDI) and cardiac fibroblasts growth medium (Cell Applications, Inc), were used. The air-saturated medium (Cal100) was medium straight from the bottle, warmed to around 37° C. And the 0% air-saturated medium (Cal0) was medium with 5% completely dissolved Na₂SO₃ and placed in the 37° C. water bath for 30 min. In testing pH's influence on oxygen readout, the media were adjusted with NaOH or HCL to pH 10 or pH 4.

### Cell Plating and Peri-Cellular Oxygen Monitoring

Human iPSC-derived cardiomyocytes (iCell Cardiomyocytes² CMC-100-012-001 from a female Caucasian donor) from Fujifilm Cellular Dynamics International (CDI), and human cardiac fibroblasts, CF (Cell Applications, Inc.) were thawed according to manufacturer's instructions. Cells were plated (50,000 cells per well) in the wells of a 96-well glass-bottom plate containing half-moon shaped oxygen sensors, that have been sterilized and fibronectin-coated (at 50 μg/ml). Culture medium exchange was done every 48 hours. In some experiments, hypoxia was induced on day five after plating by filling the wells to the top with culture medium and sealing them with oxygen-impermeable tape before readout in the VisiSense system.

The VisiSenseTD system was temperature-equilibrated in the cell culture incubator at least an hour before the start of measurements. For the whole plate oxygen monitoring in human iPSC-CMs, the peri-cellular oxygen measurements started 5 hours after the cell plating, after the switch from the cell plating medium to the cell maintenance medium. And for the hiPSC-CMs and cardiac fibroblast hypoxia comparison experiment, oxygen monitoring started right after the hypoxic condition was established. Oxygen recordings were set to continue for 24 h to 48 h, with a 10 min sampling interval and 0.8 sec exposure time.

### Cell Visualization

Cells grown on the non-transparent oxygen sensors were visualized using an upright Leica TCS SP8 microscope with a 25× water immersion objective (for this imaging, sensors were attached to the glass bottom 35 mm dishes). Cell imaging of the glass-bottom portion of the 96 wells was performed using an inverted confocal microscope (Zeiss LSM 710) or inverted Nikon Eclipse Ti2 microscope. Samples were formaldehyde-fixed, permeabilized and labeled as described previously. Nuclei were labeled with Hoechst (H3570, Thermo-Fisher Scientific), the cytoskeleton was labeled either for F-actin using Alexa-488 phalloidin (A12379, Thermo-Fisher Scientific) or using α-actinin antibody (A7811, Millipore-Sigma), and some samples were genetically modified to express the optogenetic actuator Channelrhodopsin-2 with a fluorescent reporter eYFP.

### Oxygen Data Analysis

Peri-cellular oxygen readings were acquired as PNG images and then analyzed through VisiSensVS software by selecting regions of interest in each image. Measured ratios were calibrated to percentage oxygen readings using two-point calibration with 5% Na₂SO₃ and upon saturation with ambient air. Time-dependent calibration files were applied in the first two hours of recording, considering the temperature-induced changes in transferring the plate from room temperature operation to the 37° C. humidified incubator. Whole-plate data normalization was applied by identifying the maximum and minimum ratio readouts from the whole plate throughout the whole period of recording. A scale factor was calculated by setting the maximum oxygen reading as 18.6%, the equilibrium oxygen concentration in a cell culture incubator.

### Statistical Analysis

To quantify the influence of exposure time on the ratio of red to green luminescence intensity, the mean and standard deviation (mean±SD) of ratios from a 96-well plate were computed and displayed. The spatial variation of the ratios across a plate was assessed for 0.8 s of exposure time. For time-dependent calibration and for the assessment of the effect of pH on oxygen measurements, the change in the ratio of red to green luminescence intensity over time was calculated and displayed as (mean±SD).

### Mechanism of Optical Peri-Cellular Oxygen Measurements

Optical peri-cellular oxygen measurements were based on dynamic oxygen quenching of a ruthenium dye embedded in the sensor patches on top of which cells were grown. For quantitative ratiometric readout, red and green luminescence intensity ratios were converted to percentage of oxygen saturation (pO2) using an adapted Stern-Volmer equation:

$$\frac{{R}_{o}}{R} = {( ( \frac{A}{1 + ksv * pO 2} ) + ( 1 - A ) )}^{- 1} ) \qquad ( 1 )$$

where R is the measured luminescence ratio, R₀ is the luminescence ratio at 0% O₂, ksv is the Stern-Volmer constant indicating the efficiency of oxygen quenching, and A is 0.82, a parameter for the nonlinearity of the sensing material. The ksv was computed using the two-point calibration described in the Methods, and a time-dependent ksv was applied at the beginning of the recordings to account for temperature changes. Equation 1 was linear for the limited range of oxygenation that is typical for cell cultures (<20%). However, when measured over a full range of 0-100% oxygen concentration, the inverse Stern-Volmer is expected to follow a decaying exponential function.

### High-Throughput Compatibility

The semicircular oxygen sensors covered half of each well in a 96-well glass bottom plate (FIGS. 12(c), 12(d)). This feature enabled multiparametric optical high-throughput measurements from the other half of each well. Such measurements include cellular action potentials, intracellular calcium transients, and contractility. Laser cutting of the sensors was quick and reproducible (FIG. 12(e)), where more than 96 half-moon sensors could be cut in less than 10 minutes. Subsequent attachment of the sensors in each well of a 96 well plate could be completed in less than one hour. Laser cutting had a negligible effect on sensor performance and provided sufficient sensor area for good ratiometric measurements after selecting a region of interest from each sensor. We found that it is essential to keep the pre-cut sensors in the dark and to use them within 6 months of laser cutting for best results. Sterilization of the sensors with ethanol for cellular experiments did not affect their performance.

### System Characterization and Validation

The RGB images of the entire plate (1280×1024 pixels, 24 bit) acquired by the VisiSensTD system provided sufficient contrast with approximately 1000 pixels per sensor, and 300 to 700 pixels per sensor region of interest (FIG. 12(e), middle). After calibration, the pseudocolor images of the plate clearly denoted differences in oxygenation between wells. In the example shown in FIG. 12(e), right, the quadrants of the plate were conditioned with 0% air-saturated PBS (top left), 100% air-saturated PBS (top right), room air (bottom left), and 100% air-saturated PBS with pH=4 (bottom right). We found that solution pH had negligible influence on calibrated oxygen sensor values. The oxygen sensors also reacted differently in air than when submerged in solution.

Spectral characterization of the optical oxygen sensors and validation of the oxygen measurements with Clark electrodes confirmed that small changes in peri-cellular oxygen concentration could be accurately measured in 96-well plates (FIG. 13). The Presens oxygen sensors (SF-RPsSU4) had a peak emission of 653 nm upon excitation with 409 nm light, and this emission decreased in response to increased oxygen concentration (FIG. 13(b)), demonstrating dynamic ruthenium fluorescence quenching by oxygen. Increased fluorescence in the green band (485-570 nm) was also measured from the sensors upon illumination with 409 nm light (FIG. 13(d)), with a midpoint of 510 nm. The intensity of this band did not change when oxygen concentration increased, corresponding to the oxygen insensitive reference dye that Presens has added to the sensor material. These controlled spectral measurements in a 96-well plate using our HT-μUPS system confirmed adequate spectral sensitivity of the sensors for the range of peri-cellular oxygen that is expected for cell cultures (<18.6%). The relationship of the ratio of red to green emission intensity was linear (FIG. 13(e)), as predicted by Equation 1.

A recent study cross-validated VisiSensTD peri-cellular oxygen measurements using a fiber-optics oxygen microelectrode. In contrast, we used in-line Clark electrodes to validate the measurement of peri-cellular oxygen using laser-cut semicircular sensors, the VisiSensTD system, and physiological flow rates through our HT-uUPS system in a 96-well plate. Our system demonstrated appropriate responsiveness to changes in media oxygen concentration and flow. During a 30 min recording, media flow began at 500 mL/min, was reduced to 200 mL/min, and restored to 500 mL/min. Oxygen values from the Clark electrode positioned at the inlet of the plate indicated that media oxygenation was near 100% and remained constant. Oxygen values from the Clark electrode positioned after the plate aligned with values measured optically from the sensor at the bottom of the first well. The diameter and length of tubing and the time of the peak oxygen value (85%) measured at the bottom of the well corresponded to the flow rate of 500 mL/min. The drop in oxygenation at 200 mL/min indicated a loss of oxygen as the media traveled more slowly through the connection at the entrance of the HT-uUPS cover.

### Optimal Exposure Time

Excitation light was not uniformly distributed across the bottom of 96-well plates. This increased the spatial variance (well-to-well differences) of the two dyes (e.g., red:green) emission ratios from the sensors for short excitation light exposure times. The dependence of sensor emission ratio variability on illumination exposure times between 0.15 to 1.5 secs was measured to identify exposure times where spatial variation was minimized. Spatial variation was highest for exposure times less than 0.4 sec. Spatial variation was lowest for exposure times between 0.4 and 0.9 sec. An optimal exposure time of 0.8 sec was chosen within this range as a duration for the follow up experiments. The red: green ratio for different wells of a 96-well plate for an exposure time of 0.8 sec at two levels of oxygenation (0% and 100% air-saturation) and two cell culture media (hiPSC-CMs and cardiac fibroblast) varied within ±6%. The standard deviation for hiPSC-CM media was 6% and 2%, for Cal0 and Cal100, respectively; the standard deviation for cardiac fibroblasts media was 3.5% and 1.7% for Cal0 and Cal100, respectively. This variability was much lower than the typical variability between experimental groups of cultured cells.

### Temperature Effect and Time-Dependent System Calibration

As noted, the oxygen sensors 224 (FIG. 12(c)) emit red and green colors in response to the oxygen concentration level. Temperature had a significant effect on the red: green emission ratio for each oxygen sensor, which was evident after placing a 96-well plate at room temperature in the incubator maintained at 37° C. The effect of temperature on emission ratio, as a plate was warmed to 37° C., was measured over 4 hours after placing a plate in the incubator. Wells contained media for hiPSC-CMs or media for cardiac fibroblasts, and wells had an oxygen level of either 0% or 100% air-saturation. Oxygen images were acquired every 5 minutes. The average red: green ratio for each combination of media and oxygenation changed over the first 40-90 min (40 min for the cFB media and 90 min for the hiPSC-CM media). Ratios for wells having media at 100% air-saturation were similar throughout the 4 hours and changed approximately 10% in the first hour. Ratios for wells having media at 0% changed as much as 60% in the first two hours and the change was greater for wells with fibroblast media. This result was likely due to differences in the content of the two types of media. The Stern-Volmer ksv values (Equation 1) computed from the ratios for wells having media at 0% also varied within the first 40-90 min. These results necessitated the development of a protocol to correct for the effect of changes in well temperature as a plate was warmed to 37° C. and for the effect of cell culture media content that determines media color.

### Effects of Media Color and pH

The substrate, salt, and chemical content of the hiPSC-CM media and the cardiac fibroblast media were different, resulting in each culture media having a distinct color. Media pH also determines media color and changes in pH could have an independent effect on sensor luminescence. The effect of media color and pH on sensor emission ratio was studied using 96-well plates and the VisiSensTD system. Wells contained either PBS, hiPSC-CM media, or cardiac fibroblast media and the pH of each well was set to be either 4 or 10. Differences in the color of the media in each well were clearly visible. However, color differences were not evident in images of the emission ratio. Color differences were also not evident in pseudocolor images after computing the percent oxygen concentration of each well using the emission ratios. To determine if pH had a direct effect on sensor luminescence, the emission ratio of each media having 100% air-saturation at standard pH of 7 or a pH of 4 or 10 was measured once every 5 minutes for one hour using the VisiSensTD system. No significant difference was detected between media having a pH of 7 and 4. The emission ratio was consistently lower for media having a pH of 10, which is highly alkaline and has less biological relevance for cell culture experiments.

### Long-Term Monitoring of Peri-Cellular Oxygen Concentration in Cardiac Cells

After validation, characterization, and the development of a robust calibration protocol, the VisiSensTD oxygen imaging system was used to continuously measure peri-cellular oxygen concentration from cardiac cells cultured in a 96-well plate. Using an upright microscope with a water-immersion objective, we confirmed that human iPSC-CMs formed a dense cellular syncytium on top of the oxygen sensors after coating with fibronectin. The transparent half of the glass-bottom of each well that was not covered by a sensor enabled human iPSC-CMs and human cardiac fibroblasts to be monitored to confirm healthy growth. This corroborated no adverse effects of the sensors as cells thrived as a syncytium equally well on top of each sensor and on the glass-bottom portion of the well that was not covered by a sensor. In monitoring cell function over several days, we confirmed that sensor readouts were stable and did not alter cell growth.

Typical images of human iPSC-CMs and human cFBs grown on fibronectin-coated glass-bottom portion. Studies were conducted for time intervals ranging from 24 to 48 hours. In one experiment, peri-cellular oxygen was measured every 10 minutes over 48 hours from hiPSC-CMs plated in every other row of a 96-well plate. Two control wells had oxygen sensors and media but no cells. Peri-cellular oxygen dropped dramatically to <5% within 10 hours in all wells that had cells. Oxygen concentration in control wells was constant at 18.6%, corresponding to the equilibration of the media with the incubator oxygen concentration. In wells having cells, two patterns of peri-cellular oxygen dynamics emerged after 10 hours: one of monotonic oxygen depletion and another of intermittent depletion. In cases of monotonic depletion, after the initial oxygen drop to <5%, peri-cellular oxygen monotonically and slowly decreased to a steady-state level. In intermittent depletion, peri-cellular oxygen oscillated intermittently after the initial oxygen drop. For the results, monotonic depletion was observed in 11 out of 38 wells (29%) and intermittent depletion occurred in 27 out of 38 wells (71%). Oscillatory intermittent depletion occurred in different wells within the rows of a plate, confirming that oxygen dynamics were independent of the position of a well within a plate.

Dynamic patterns of peri-cellular oxygen were studied in cardiac fibroblast and hiPSC-CM cultures over 24 hours to determine if depletion occurs more rapidly in cells having higher metabolic rate. Depletion was hypothesized to occur more rapidly in hiPSC-CM cultures due to the higher oxygen consumption of contracting cells. The two cell types were cultured in separate sets of 6 wells of the same plate. Two of those wells were filled with the typical 200 mL of media to provide normoxia while the other four wells were filled with 300 mL of media and sealed with oxygen-impermeable tape to generate hypoxia. Peri-cellular oxygen was depleted within 6 hours for normoxic and hypoxic cultures of hiPSC-CMs. Oxygen depletion was much slower for fibroblasts, where over 24 hours oxygen did not drop below 10% for normoxic cultures and most hypoxic cultures maintained an oxygen level above 5%. These results confirm the hypothesis and also demonstrate the utility of optical oxygen sensing in providing long-term measurements that reveal cell-type differences in the baseline and fluctuations of peri-cellular oxygen concentration.

### Discussion

Oxygen consumption and defense mechanisms against hypo-/hyperoxia are critical to sustaining life, as O₂ is a key component of energy (ATP) production in the mitochondria. However, when in excess, oxygen is inherently toxic due to its chemical reactivity and the generation of reactive oxygen species (ROS). Although the need for measuring peri-cellular oxygen levels in cell culture has been recognized since at least 1970, doing so is not routine in cell culture studies.

Recent developments have included scalable solutions to control dissolved (bulk) oxygen levels in custom-designed 96-well plates. For example, in one study the well bottoms of a 96-well plate were modified to use special actuation posts, driven by magnetic field to increase oxygen availability. In other studies, elegant microfluidics-based solutions with concentration gradients have been deployed to control dissolved oxygen levels in custom-designed 96-well plates with oxygen-permeable bottom. Fiber-optic probe was used to perform sequential measurements in different wells and confirm oxygen levels close to the well bottom, which for cells with low metabolic needs may approach peri-cellular oxygen levels.

In bacterial cell culture applications, more advanced high-throughput monitoring solutions have been implemented. For example, oxygen-sensing fluorophores have been embedded in hydrogel inserts to monitor dissolved oxygen levels in custom 96-well plates up to several hours. Another bacterial cell culture study monitored peri-cellular oxygen by methods similar to the ones reported here, including the use of a commercial 96-well OxoPlate by Presens. In one variant, circular sensor patches were cut and glued in standard 96-well plates; a robotic arm was moving the plate between a shaker and a plate reader to track oxygen levels as function of shaking speeds to optimize microbial culture conditions. Because of the discontinuous readouts, the study purposefully sought to develop oxygen sensors with slower response time than the ones deployed here. OxoPlate (Presens) was also used to study antibiotic resistance in microbial cell culture based on longitudinal tracking of peri-cellular oxygen.

Overall, high-throughput peri-cellular oxygen measurements in mammalian cell culture are rare, and no long-term measurements are available from cultures of human iPSC-CMs. A major obstacle has been the lack of user-friendly monitoring systems and reliable non-invasive methods for long-term oxygen monitoring in common cell culture formats such as 96-well plates.

In this study, the Presens ratiometric optical oxygen sensors and camera-based imaging system were adopted to track peri-cellular oxygen dynamics in human cardiac cells cultured in glass-bottom 96-well plates. We developed a scalable technique for reproducible cutting and positioning of semicircular optical oxygen sensors into glass-bottom HT plates, to cover half of each well, leaving the other half for multimodal structural and functional imaging. We validated the optical oxygen readings with traditional Clark electrodes positioned in line in bulk measurements and during perfusion within 96 wells using our specialized microfluidics-based cover. The spectral, spatial, and temporal properties of the system were systematically characterized. Optimization of the exposure time and the two-point calibration allowed continuous oxygen measurements in a standard incubator that were robust and reliable.

Compared to prior studies, the following new and useful aspects are demonstrated here: 1) to our knowledge, this is the first mammalian culture study to track long-term peri-cellular oxygen in standard glass-bottom 96-well plates, that are the industry standard and are used in preclinical testing; 2) the first study to do so in human cardiomyocytes over long term, with continuous frequent sampling of peri-cellular oxygen levels during cell growth in a standard cell culture incubator, providing novel insights on cardiomyocyte oxygen consumption dynamics; 3) our approach in using half of each well for optical oxygen sensing (semicircular sensors) allows multimodal structural and functional observations in the same samples through the transparent glass bottom part, compatible with standard microscopy and with all-optical electrophysiology systems.

Our results are in agreement with in vivo estimates of peri-cellular oxygen levels in the heart, where oxygen tension in the immediate vicinity of the cardiomyocytes is in the range of 3-6%. Namely, for human iPSC-CMs, in the static culture conditions of a glass-bottom 96-well plate, we observed a drop to <5% oxygen within several hours of media exchange. After this initial drop, the cells either settled to a steady state of low pericellular oxygen, likely due to the balance between active oxygen consumption and passive diffusion, or exhibited an intermittent oxygen dynamics. These two types of responses are interesting novel observations and require further investigation. In contrast to the cardiomyocytes, the non-contracting cardiac fibroblasts exhibited higher peri-cellular oxygen levels after dropping to a stable steady-state, as expected for cells with lower oxygen demand.

In vivo, cardiac cell normoxia is maintained by feedback mechanisms that tightly regulate coronary blood and by oxygen-on-demand provided by the excellent oxygen carrying capacity of hemoglobin. Ex vivo, in the absence of adequate oxygen buffering, perfused working hearts experience hypoxic conditions. In vitro, cardiomyocytes may experience hyperoxic, normoxic or hypoxic conditions depending on their density, electromechanical activity, mass transport conditions and the shortest path to the ambient oxygen supply. For human iPSC-CMs, oxygen tension was recognized in early work as a key variable for optimizing cellular differentiation and maturation; transient control of oxygen level is used during the differentiation of iPS cells into cardiomyocytes. Hypoxia signaling is also a foundational physiologic component of mature cardiomyocytes, where it intimately regulates electromechanical function, including ion channel currents and protein expression. Based on this, longitudinal label-free monitoring of peri-cellular oxygen, in a high-throughput manner, provides unique insights into the metabolic state of the cells, and potentially can be correlated with their level of maturation.

The system described here is best suited for imaging the oxygen concentration of two-dimensional multi-cell structures, such as monolayers. This is a limitation, considering the growing popularity of three-dimensional cell constructs, including cell spheroids and microtissues. A potential way to extend the described label-free oxygen sensing approach to 3D structures is inspired by the recent work of others, where optical sensors have been mounted on transparent prisms and oxygen-sensing cell culture vessels have been thermoform-molded to line the wells of spheroidal plates. Future work includes automating the placement of sensors into 96-well plates using the scalable technique described and manually demonstrated here. Combination of the high-throughput monitoring of peri-cellular oxygen with microfluidics-based solutions of controlling oxygenation can yield feedback-controlled growth environment for cardiac tissue engineering. Coupling label-free measurements of peri-cellular oxygen with label-free measurements of cardiac electromechanical waves will also provide valuable insights into the interplay between cellular activity and oxygenation state.

As shown in FIG. 1(c), one or more processing devices 210 are configured to implement the system and method of the present disclosure. It is noted that the processing device can be any suitable device, such as a computer, server, mainframe, processor, microprocessor, controller, PC, tablet, smartphone, or the like. The processing device can configured in combination with other suitable components, such as a display device (monitor, LED screen, digital screen, etc.), memory or storage device, input device (touchscreen, keyboard, pointing device such as a mouse), wireless module (for RF, Bluetooth, infrared, WiFi, etc.). The information may be stored on a computer medium such as a computer hard drive, on a CD ROM disk or on any other appropriate data storage device, which can be located at or in communication with the processing device. The processing device is configured to conduct the entire process automatically, and without any manual interaction. Accordingly, unless indicated otherwise the process can occur substantially in real-time without any delays or manual action.

### FURTHER EMBODIMENTS

Several improvements to this technology may be considered. The material choice for the cover of is not limited to PMMA, other materials such as Polystyrene (PS), Cyclic Olefin Copolymer (COC), Polydimethylsiloxane (PDMS, a silicone), Polycarbonate (PC) among others can also be used. While methods of ethylene oxide and hydrogen peroxide have been demonstrated effective without harming the cover material, further testing is required to ensure repeatability. Furthermore, the non-uniform shear condition across the bottom can be addressed by altering the geometry and flow path of the well-fitting. Such shear effects can be simulated (e.g. using COMSOL Multiphysics or ANSYS) by first modeling alternate geometries that modify the flow path within the chamber to achieve a more fully developed flow. These geometries can be simulated in COMSOL at different plug heights and flow rates to determine an optimal shear stress uniformity. Additionally, longer-term studies can be performed when investigating the roles of oxygen delivery, nutrient delivery, and shear stress on biofilm biomass. Lastly, the system would benefit from a precise gas control as the availability of oxygen has been shown to affect bacterial biofilm growth.

To adapt to situations requiring oxygen control, the system can use two strategies. The first would be to add active oxygenation to the reservoir to prevent depletion while using closed loop recirculating flow. The second strategy involves modification of the perfusion plug length to alter oxygen delivery time. This delivery time can be further optimized while considering the bottom shear stress through simulation modeling. The following sections will discuss design improvements made to shear stress distribution and system priming.

Aside from its potential as a biofilm reactor, this technology may find additional applications in antibiotic susceptibility testing, extracellular polymeric substance biogenesis, pharmacokinetic and pharmacodynamic studies, biomanufacturing, drug testing on cultured tissue slices, organs-on-chip and basic biomedical research. Each of these areas may benefit from the availability of automated perfusion with well controlled flow and optical evaluation in situ.

### System Design

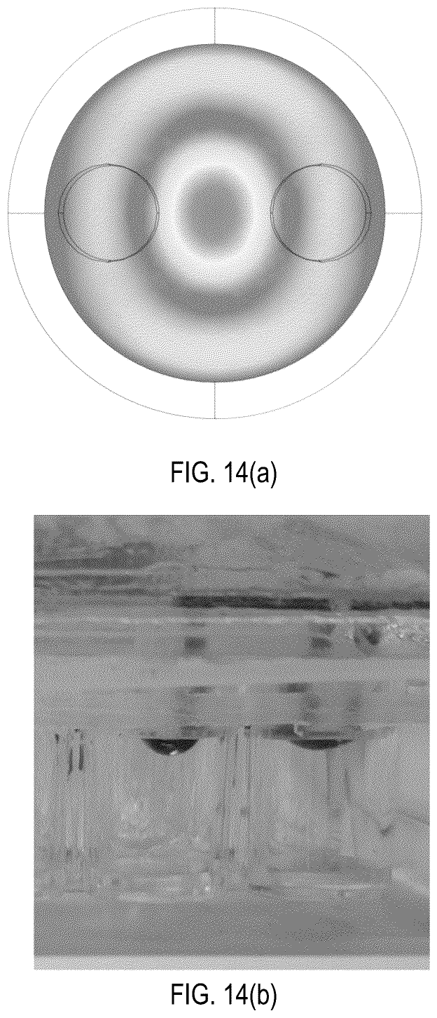

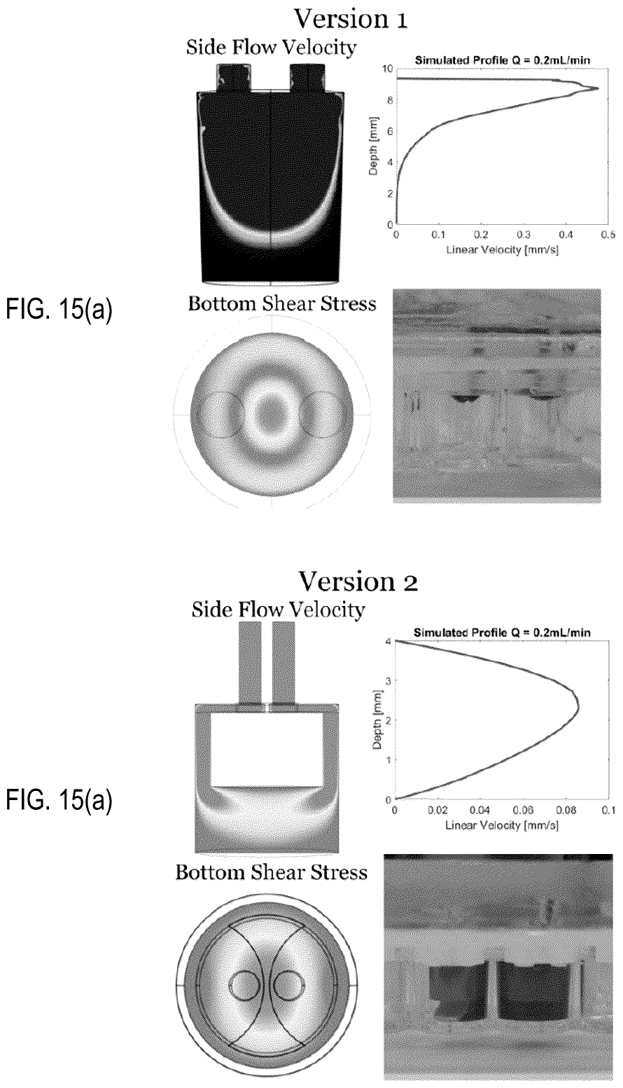

Referring to FIG. 14(a), among the identified problems with the first embodiment of the system (FIG. 1(b)) were well plate priming and simulated bottom shear stress. Both problems could be addressed by redesigning the cover and this section will cover the redesign. FIG. 15 shows a Single Well side view schematic of version 1 (FIG. 1(b)) and version 2 (FIG. 2(a)) covers. A key difference between the versions is the flow path that wraps around the side of the well which restricts the movement to follow more closely flow within a channel.

The central idea behind the redesign was to restrict the flow path to behave more like a flow channel over a large portion of the well bottom. The design shown in FIG. 2(a) was developed as a result. This new version (FIG. 2(a)) restricts flow to the perimeter of the well and forces a more unidirectional flow path along the bottom of the well. This design does increase fabrication complexity, however, and this factor must be considered when pushing for a more idealized flow geometry. The new version 2 (FIG. 2(a)) requires two additional CNC milling steps and an additional thermal bonding step before fabrication is completed for hard plastic materials such as PMMA (acrylic).

Numerical simulation in COMSOL suggested that the version 2 side flow was both more fully developed (parabolic) and that bottom shear stress was more uniform (FIGS. 15(a), (b)). Furthermore, the priming was improved in that version 2 did not require the wells to be pre-filled for priming. However, it is worth noting the significant liquid column height differences between versions and investigating whether this factor alone would improve priming. However, this factor alone is not sufficient to improve shear stress uniformity as will be demonstrated in the following sections.

### Characterization of Flow Velocity Profile

To verify the simulation of the redesign, a particle tracing experiment was performed. Briefly, 3 μm fluorescent beads were dissolved in excess of pure water and flowed through the Version 2 cover of the system at 0.25 mL/min. 15 seconds of time lapse images were captured at 30 Frames per second. Images were taken with an Olympus X71 fluorescent microscope with CCD camera and 10× objective. The z focal plane was manually adjusted with the fine adjustment to sweep through different points along the flow path at the well center. Particle tracing was performed in Matlab modified routines from, and results were plotted against the simulated results along the center profile.

The particle tracing result shows good agreement between experimental and simulated conditions and thus numerical simulation was relied upon for further optimization of the shear stress distribution.

Design and Fabrication of Even Shear Cover with Varying Liquid Column Heights

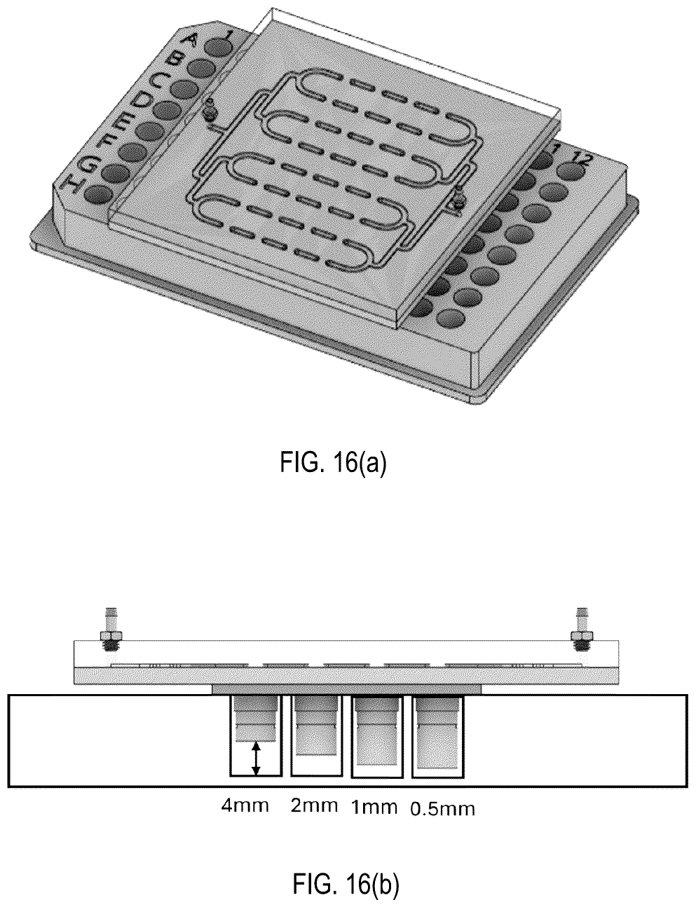

With these simulations results suggesting that version 2 provided superior shear stress uniformity, a second bacterial biofilm study was conducted investigating the role of different shear stress conditions over 7 days of perfusion. Notable changes to the design of the cover include the parallel replicates along well plate columns (FIG. 16(a)) and the varying liquid column heights (and therefore shear stress) in series (FIG. 16(b)). This cover was fabricated with similar materials to version 1 using PMMA hard components and a soft PDMS gasket.

The system design was altered for this 7 day perfusion experiment in part due to the concern of recirculating flow. While recirculating flow drastically reduces media volume requirements, this flow method raises questions of contamination and sample independence, particularly as the bacterial population grows within suspension. Clogging is also a concern and as a result, a syringe pump was used that could provide greater pressure. The syringe pump was set up outside the incubator with media, well plate, and waste reservoirs inside the incubator. A check valve circuit was connected in a T shape at the outlet of the syringe to allow the system to be automated. A Matlab script was used to control the NE-1600 syringe pump on a 75% dispensing 25% refilling duty cycle. The total flow rate was set to 2 mL/min so that flow within a given well was 250 μL/min. Higher flow rate may be preferred, but this provided a reasonable compromise between experiment perfusion time, reagent volume required, and flow rate. Overall, a total of 15.1 L was required for the seven-day perfusion.

### Results of Seven Day Biofilm Perfusion

E. Coli were plated onto 96 well plates. Again, a static culture was performed as control. To quantify biofilm matrix morphology at the well plate bottom, a staining protocol was used to prepare Congo red and Syto 9 solutions. Following 7 day perfusion, the cover was removed, and all samples were rinsed with pure water before staining with Syto 9 and Congo Red. These dyes were used to identify bacterial cells and the amyloid protein curli. Imaging was conducted on a spinning disk confocal microscope (FEI CorrSight) and image analysis was performed with Fiji. First, single 2 D images were collected in widefield view with a 5× objective on just the green channel to compare surface coverage of well bottoms. Selected images. To quantify the surface coverage, the threshold selection in Fiji was used to binarize each image with Otsu's method and then to define a circular region within which to calculate the area fraction of pixels. Each perfusion condition had significantly more surface coverage than the static, the differences between perfused conditions were much less pronounced.

Furthermore, images were taken in spinning-disk mode with a 20× objective to obtain z-stacks of both green and red channels. These image stacks were processed in Fiji to develop the 3D reconstructed images. These images show some of the complex heterogeneity of biofilm growth at the well bottom. These results may also be quantified, and two methods of quantification were employed including maximum thickness and volume. To analyze the images for either maximum thickness or volume, first an in-focus image was manually selected within the stack. Thresholding was performed using Otsu's method and the threshold determined for the in-focus image was applied to the entire stack. This was performed for both green and red channels as these channels were analyzed independently. The voxel dimensions were set to 0.327 μm×0.327 μm×1 m as this was the setting used for acquisition. Next, the 3D Suite plugin for Fiji was used to first segment the biofilms with simple segmentation and a minimum size of 10 units.

Finally, the segmented image was imported to the 3D manager within the same plugin and 3D measure was selected which computes the Zmin and Zmax and volume for all segmented objects. The tables generated for each image were exported to Matlab where they could be sorted and processed to determine maximum thickness as the max (Zmax−Zmin) for each stack and volume was determined by the average of the top 5 volumes for each segment in a stack. While much focus has been put into the quantification of biofilm parameters from image analysis, the methods of image analysis may significantly influence results and further experiments would be needed to validate this protocol. The results suggest that biofilm maximum thickness and volume increase with shear stress and that they show the most difference when compared to the static control.

It was generally observed that the addition of perfusion improved biofilm surface coverage on the well bottom when compared to a static assay, but it was not clear that once covered, additional perfusion promotes further growth.

Oxygen consumption of hiPSC-CMs has not been studied in high-throughput (HT) format plates used in pre-clinical studies. This chapter discusses a comprehensive characterization and validation of a system for HT long-term optical measurements of peri-cellular oxygen in cardiac syncytia (human iPSC-CM and human cardiac fibroblasts), grown in glass-bottom 96-well plates.

Temporal Characterization with Perfusion Cover

The responsiveness of the oxygen sensing system to changes in media oxygen concentration and flow rate was characterized using 96-well plates and a new oxygen impermeant version of the HT-uUPS cover described in our previous work. This cover was assembled in two components, one soft PDMS sealing gasket and one acrylic perfusion base that was CNC-milled from cast acrylic (McMaster-Carr 8560K354) and thermally bonded using a heat press (Rosineer Grip Twist) at 130° C. for 3 hours. The sealing gasket was fabricated by pouring a mixture of Sylgard 184 (Dow) and Dragonskin 10 (Smooth-On) in a 1:2 ratio into an acrylic mold. Finally, an inlet and outlet were tapped (10-32 thread) for each well and fitted with stainless steel 10-32 barb to-thread connectors (Pneumadyne).

The sensor response within the perfusion system HT-uUPS was validated. Clark electrodes were placed before and after the cover. An optical oxygen sensor was placed at the bottom of the leftmost well of the cover. The Clark electrodes and VisiSensTD system were calibrated with water at 100% O2 and 0% O2. Water bubbled with 100% O2 was loaded into a syringe pump and perfused through the cover. Flow rate was measured using a flow meter (Sensirion SLI-2000) and varied between 500 μL/min and 200 μL/min to determine the system's response to changes in flow.

Validation of the oxygen measurements with Clark electrodes confirmed that small changes in peri-cellular oxygen concentration could be accurately measured in 96-well plates. A recent study cross-validated VisiSensTD peri-cellular oxygen measurements using a fiber-optics oxygen microelectrode. In contrast, in-line clark electrodes were used to validate measurement of peri-cellular oxygen using laser-cut semicircular sensors, the VisiSensTD system, and physiological flow rates through our HT-μUPS system in a 96-well plate. Our system demonstrated appropriate responsiveness to changes in media oxygen concentration and flow. During a 30 min recording, media flow began at 500 μL/min, was reduced to 200 μL/min, and restored to 500 μL/min. Oxygen values from the Clark electrode positioned at the inlet of the plate indicated that media oxygenation was near 100% and remained constant. Oxygen values from the Clark electrode positioned after the plate aligned with values measured optically from the sensor at the bottom of the first well. The diameter and length of tubing and the time of the peak oxygen value (85%) measured at the bottom of the well corresponded to the flow rate of 500 μL/min. The drop in oxygenation at 200 μL/min indicated a loss of oxygen as the media traveled more slowly through the connection at the entrance of the HT-uUPS cover.

### Methods of Dynamic Oxygen Monitoring and Analysis

### Methods of Cell Plating and Pericellular Oxygen Monitoring

Human iPSC-derived cardiomyocytes (iCell Cardiomyocytes CMC-100-012-001 from a female Caucasian donor) from Fujifilm Cellular Dynamics International (CDI), and human cardiac fibroblasts, CF (Cell Applications, Inc.) were thawed according to manufacturer's instructions. Cells were plated (50,000 cells per well) in the wells of a 96-well glass-bottom plate containing half-moon shaped oxygen sensors, that have been sterilized and fibronectin-coated (at 50 μg/ml). Culture medium exchange was done every 48 hours. In some experiments, hypoxia was induced on day five after plating by filling the wells to the top with culture medium and sealing them with oxygen impermeable tape before readout in the VisiSense system.

The VisiSensTD system was temperature-equilibrated in the cell culture incubator at least an hour before the start of measurements. For the whole plate oxygen monitoring in human iPSC-CMs, the peri-cellular oxygen measurements started 5 hours after the cell plating, after the switch from the cell plating medium to the cell maintenance medium. And for the hiPSC-CMs and cardiac fibroblast hypoxia comparison experiment, oxygen monitoring started right after the hypoxic condition was established. Oxygen recordings were set to continue for 24 h to 48 h, with a 10 min sampling interval and 0.8 sec exposure time.

### Oxygen Data Analysis

Peri-cellular oxygen readings were acquired as PNG images and then analyzed through VisiSensVS software by selecting regions of interest in each image. Measured ratios were calibrated to percentage oxygen readings using two-point calibration with 5% Na2SO3 and upon saturation with ambient air. Time-dependent calibration files were applied in the first two hours of recording, considering the temperature-induced changes in transferring the plate from room temperature operation to the 37° C. humidified incubator. Whole-plate data normalization was applied by identifying the maximum and minimum ratio readouts from the whole plate throughout the whole period of recording. A scale factor was calculated by setting the maximum oxygen reading as 18.6%, the equilibrium oxygen concentration in a cell culture incubator.

### System Calibration

A two-point calibration converted the ratio of red to green luminescence intensity imaged from the sensors into percentage of air saturation. The terms ‘Cal 0’ and ‘Cal 100’ were used in system characterization experiments for 0% and 100% air saturation, and oxygen saturation levels ranging from 0% to 18.6% were displayed in cell experiments (the equilibrium oxygen partial pressure in a humidified 37.C, 5% CO2 incubator is 18.6%).

Two types of culture media, CDI iCell Cardiomyocytes maintenance medium (Fujifilm CDI) and cardiac fibroblasts growth medium (Cell Applications, Inc), were used. the air-saturated medium (Cal 100) was medium straight from the bottle, warmed to around 37.c. and the 0% air-saturated medium (Cal 0) was medium with 5% completely dissolved Na2SO3 and placed in the 37.c water bath for 30 min. In testing pH's influence on oxygen readout, the media were adjusted with NaOH or HCl to pH 10 or pH 4.

### Spatial Effects and Exposure Time

Excitation light was not uniformly distributed across the bottom of 96-well plates. This increased the spatial variance (well-to-well differences) of red: green emission ratios from the sensors for short excitation light exposure times. The dependence of sensor emission ratio variability on illumination exposure times between 0.15 to 1.5 secs was measured to identify exposure times where spatial variation was minimized. Spatial variation was highest for exposure times less than 0.4 sec. Spatial variation was lowest for exposure times between 0.4 and 0.9 sec. An optimal exposure time of 0.8 sec was chosen within this range as a duration for the follow up experiments. The red: green ratio for wells of a 96-well plate for an exposure time of 0.8 sec at two levels of oxygenation (0% and 100% air-saturation) and two cell culture media (hiPSC-CMs and cardiac fibroblast) varied within +/−6%, which was much lower than the typical variability between experimental groups of cultured cells.

### Temperature Effect

Temperature had a significant effect on the red: green emission ratio for each sensor, which was evident after placing a 96-well plate at room temperature in the incubator maintained at 37° C. The effect of temperature on emission ratio, as a plate was warmed to 37° C., was measured over 4 hours after placing a plate in the incubator. Wells contained media for hiPSC-CMs or media for cardiac fibroblasts, and wells had an oxygen level of either 0% or 100% air-saturation. Oxygen images were acquired every 5 minutes. The average red: green ratio for each combination of media and oxygenation changed over the first hour. Ratios for wells having media at 100% air-saturation were similar throughout the 4 hours and changed approximately 10% in the first hour. Ratios for wells having media at 0% changed as much as 60% in the first two hours and the change was greater for wells with fibroblast media. This result was likely due to differences in the content of the two types of media. The Stern-Volmer ksv values computed from the ratios for wells having media at 0% also varied within the first hour. These results necessitated the development of a protocol to correct for the effect of changes in well temperature as a plate was warmed to 37° C. and for the effect of cell culture media content that determines media color.

Time-dependent calibration, effects of pH and medium color on the oxygen readings, a Time-dependent calibration over 4 hours, done in culture medium for human cFBs and human iPSC-CMs in a 37° C. cell culture incubator, n=96 wells. Over the first 40 min, there is a temperature dependent rise in the ratio reading for the Cal0 values. Inset shows that the derived Stern-Volmer coefficient, ksv, varies in this initial period for both culture media, b Time-dependent calibration changes of CM medium with different pH=4, 7.4 (standard) and 10. Oxygen readouts were continuously recorded for an hour in 5 min intervals. The mean ratio of air-saturated CM medium with standard and altered pH was plotted over time (mean±SE, n=12-24 samples), c Same as c, but for cFB culture medium, d Full 96-well plate characterization of medium color and pH's influence on oxygen reading. Conditions are listed in the plate layout. Although pH visibly altered culture medium color (bottom-up view), the analyzed oxygen data were not influenced by medium color or pH difference.

### Effects of Media Color and pH

The substrate, salt, and chemical content of the hiPSC-CM media and the cardiac fibroblast media was different, resulting in each culture media having a distinct color. Media pH also determines media color and changes in pH could have an independent effect on sensor luminescence. The effect of media color and pH on sensor emission ratio was studied using 96-well plates and the VisiSensTD system. Wells contained either PBS, hiPSC-CM media, or cardiac fibroblast media and the pH of each well was set to be either 4 or 10. Differences in the color of the media in each well was clearly visible. However, color differences were not evident in images of the emission ratio. Color differences were also not evident in pseudocolor images after computing the percent oxygen concentration of each well using the emission ratios. To determine if pH had a direct effect on sensor luminescence, emission ratio of each media having 100% air-saturation at standard pH of 7 or a pH of 4 or 10 was measured once every 5 minutes for one hour using the VisiSensTD system. No significant difference was detected between media having a pH of 7 and 4. The emission ratio was consistently lower for media having a pH of 10, which is highly alkaline and has less biological relevance for cell culture experiments.

### Long-Term Pericellular Oxygen Monitoring of Statically Cultured Cells

After validation, characterization, and developing a robust calibration protocol, the VisiSensTD oxygen imaging system was used to continuously measure peri-cellular oxygen concentration from cardiac cells cultured in a 96-well plate. Studies were conducted for time intervals ranging from 24 to 48 hours. In one experiment, peri-cellular oxygen was measured every 10 minutes over 48 hours from hiPSC-CMs plated in every other row of a 96-well plate. Two control wells had oxygen sensors and media but no cells. Peri-cellular oxygen dropped dramatically to <5% within 10 hours in all wells that had cells. Oxygen concentration in control wells was constant at 18.6%, corresponding to equilibration of the media with the incubator oxygen concentration. In wells having cells, two patterns of peri-cellular oxygen dynamics emerged after 10 hours: one of monotonic oxygen depletion and another of intermittent depletion. In cases of monotonic depletion, after the initial oxygen drop to <5%, peri-cellular oxygen monotonically and slowly decreased to a steady-state level. In intermittent depletion, peri-cellular oxygen oscillated intermittently after the initial oxygen drop. Monotonic depletion was observed in 11 out of 38 wells (29%) and intermittent depletion occurred in 27 out of 38 wells (71%). Oscillatory intermittent depletion occurred in different wells within the rows of a plate, confirming that oxygen dynamics were independent of the position of a well within a plate.

Oxygen dynamic patterns were studied in cardiac fibroblast and hiPSC-CM cultures over 24 hours to determine if depletion occurs more rapidly in cells having higher metabolic rate. Depletion was hypothesized to occur more rapidly in hiPSC CM cultures due to the higher oxygen consumption of contracting cells. The two cell types were cultured in separate sets of 6 wells of the same plate. Two of those wells were filled with the typical 200 μL of media to provide normoxia while the other four wells were filled with 300 μL of media and sealed with oxygen-impermeable tape to generate hypoxia. Peri-cellular oxygen was depleted within 6 hours for normoxic and hypoxic cultures of hiPSC-CMs. Oxygen depletion was much slower for fibroblasts, where over 24 hours oxygen did not drop below 10% for normoxic cultures and most hypoxic cultures maintained an oxygen level above 5%. These results confirm the hypothesis and also demonstrate the utility of optical oxygen sensing in providing long-term measurements that reveal cell-type differences in the baseline and fluctuations of peri-cellular oxygen concentration.

Optical measurements of peri-cellular oxygen in human iPSC-cardiomyocytes and human cardiac fibroblasts, cFBs, a Continuous optical sensing of peri-cellular O2 in human iPSC CMs over 48 h. Peri-cellular oxygen levels drop to <5% in <10 h for most samples. Two types of hypoxia responses are seen: (1) monotonic O2 depletion to a steady-state, and (2) intermittent O2 dynamics after an O2 dip below 5% (black arrows). Type 1 was observed in about 30% of the samples in this plate, while type 2 was observed in the remaining 70% of the measured n=40 wells. 18.6% oxygenation corresponds to the maximum (100%) oxygen saturation in a CO2 incubator, Example records of hiPSC-CMs and cFB peri-cellular O2 over 24 h, under normoxic and hypoxic conditions. Peri-cellular O2 in cFBs decreases slower and stabilizes to a higher level; induced hypoxia (filling the wells with solution to the top and sealing them) sped up O₂ the depletion in cFB. The hiPSC-CMs showed rapid depletion of O2 within 6 h with faster initial phase of drop in the sealed samples, which then bounced back to slightly higher steady-state values.

### Modeling Bioreactor Shear Stress, Oxygen Delivery, and Oxygen Demand

Bioreactors offer a controlled environment for cell culture, tissue engineering, and disease modeling, among other applications. In the case of perfusion bioreactors, the rate of flow of media is a key parameter for controlling the microenvironment, and in the case of microfluidic geometries channel height is another important feature. These design inputs should ideally be guided by knowledge of a target pericellular oxygen and bottom shear stress range. Computational fluid dynamics solvers such as COMSOL Multiphysics provide environments to quickly test and measure such parameters as pericellular oxygen and bottom shear stress which can be particularly useful when the flow channel geometry is not standard and an analytical solution to the Navier-Stokes equation is not guaranteed. These simulations can be done iteratively while varying design inputs and probing locations in space and time to determine steady state and transient values. However, these simulations can be computationally expensive and may require expertise to set up and run effectively. As such, a mathematical model that provides at least an order of magnitude estimate of bioreactor flow rate and channel height required to achieve a target pericellular and oxygen and bottom shear stress is highly desirable. In the following chapter, a theoretical framework will be introduced with the goal of characterizing bioreactor well bottom shear stress, oxygen delivery, and oxygen consumption with a set of analytical equations and dimensionless inputs. The framework begins with an assessment of the static well condition involving pure diffusion and consumption. The framework then builds on these concepts to incorporate flow within a micro perfusion channel. Finally, the framework introduces cellular energetics as design inputs tied to cellular workload as a function of oxygen consumption rate.

Framework of static well plate oxygen transport and consumption. 2D model illustration used for mathematical model derivation. Diffusion is uniform and 1 dimensional along well depth. 3D model used for numerical simulation. Diffusion remains uniform along well depth and is essentially 1 dimensional.

### Numerical Simulation Setup

Varying culture medium column heights were simulated at a given oxygen consumption rate Vmax. Time dependent simulations and parameter estimations were performed using COMSOL Multiphysics 5.5 software with LiveLink for MATLAB, and the simulation results were analyzed using MATLAB R2021b. The use of LiveLink allows for the scripting of COMSOL with MATLAB and automates the any postprocessing steps required for data visualization. The 3D model used in the simulation was designed based on the dimensions of a single well in a standard 96-well plate when filled with media to a particular volume.

To study the relationship between the liquid column height and the maximum cellular consumption of oxygen, transport of oxygen was simulated through different liquid column heights ranging from 1 mm to 9.9 mm (300 μL). Each liquid column was initially set as fully oxygenated at 179 μmol/L. Cell densities density and maximum oxygen consumption were held constant. The simulation was constructed to model both oxygen transport through the well and oxygen consumption at the bottom of the well. The oxygen transport equations are based on mass balance and molecular diffusion equations for the transport of diluted species module in COMSOL. the reaction term R is zero throughout the well volume except at the well bottom surface, where the specific boundary condition for a surface reaction is enforced. The boundary condition at the air-liquid interface was set as a constant concentration of 179 μmol/L (oxygen solubility in media at 37° C. Furthermore, an adjusted diffusion coefficient of oxygen in culture media at 37° C. was used. The mesh was generated using the default normal setting with minimum element size ranging from 0.109 mm to 0.178 mm. Minimum element quality ranged between 0.247 and 0.2841. Degrees of Freedom ranged between 4,279 and 6,469. The simulations were done on an Intel® 64 bit CPU (11th Gen Intel® Core™ i7-1165G7 @ 2.80 GHz, 2803 Mhz, 4 Cores, 8 Logical Processors with 16 G RAM) running a Windows® 11 operating system.

### Results of Model Comparison

Results are for the case of constant consumption and cell density over a sweeping liquid column height annotated by the liquid volume at that height (50, 100, 200, and 30 μL). It is important to note that the Damkohler number may be affected by several parameters and does not uniquely define a pericellular oxygen for a given parameter (height, consumption rate, etc) unless others are held constant. The case for a typical workload of cardiomyocytes when cultured under different liquid column heights. For this case, we may define a normoxic region greater than 1% and less than 7% oxygen which corresponds to theta greater than 0.065 and less than 0.37. The results show that for this cellular workload, the steady state normoxic region may be achieved by culturing with a liquid column height between 100 and 200 μL.

Plot of mathematical and simulate results for nondimensionalized static pericellular oxygen. Cellular oxygen consumption rate is held constant to investigate the sweep of liquid column height. Different column height volumes are shown with their corresponding Damkohler numbers. The normoxic region for this case is annotated with horizontal dashed lines and occurs roughly between 100 μL and 200 μL column heights.

### Numerical Simulation Setup

A modified design was drawn in Fusion 360 and imported to COMSOL for simulation. This geometry was drawn to mimic microchannel perfusion along the highlighted region. Oxygen was evaluated along the well bottom at steady state. To reach steady state, simulations were run as time dependent studies for 24 hours and plotted to visually determine that the model had reached steady state. A total of 43 conditions were simulated. Please see Appendix B for the full table of simulation parameters and results. The following sections will go over selected results of simulation and mathematical modeling to emphasize the utility and limitations of the mathematical approach. Of note, the total time required to simulate these conditions was approximately 9 hours while the mathematical model was finished in approximately two minutes for the same number of conditions. While the numerical simulation considers more spatial and temporal factors, it is also more expensive.

The Damkohler number in the mathematical model does not change as the concentration approaches Km whereas the simulated model uses the full Michaelis-Menten equation to inform reaction kinetics. As a result, the mathematical model may reach a zero pericellular oxygen concentration before the numerical model.

The results of comparing simulation with the mathematical model. The model agreement is worse when steady state oxygen is low which occurs for higher cellular workloads. Varying cellular workload parameters for both the simulated and mathematical models. Results are for pericellular oxygen at steady state (t=24 hr) along center flow path for the same cover geometry (H=0.5 mm), and flow rate (Q=1 μL/min). Model agreement is good provided steady-state oxygen concentration is sufficiently high.

### Considering Bioreactor Design

The potential of the theoretical model to inform bioreactor design parameters of liquid column height and flow rate. Results of mathematical calculations for shear stress and pericellular oxygen activity for a moderate cellular workload at the center of the well. These results show the potential to quickly sweep over a large range of design inputs. From these results, a critical Peclet number may be observed at 0.97 where below this number, the pericellular oxygen drops significantly. Furthermore, if the design is restricted to a maximum shear stress of 1 mPa, then the highlighted region shows preferred values that could be further tuned with numerical simulation or used as an order of magnitude estimate if numerical simulation is not available. Therefore, this model may be used to inform the perfusion bioreactor design based on the four dimensionless parameters previously discussed.

Heatmap of mathematical model results of steady state oxygen and shear stress when sweeping through channel Peclet number and geometric factors. The Damkohler number is also shown for reference but is strictly a function of channel height for this case as the oxygen consumption rate is held constant. Highlighted regions in the graphs show critical Peclet and shear values to consider for bioreactor design.

It will be apparent to those skilled in the art having the benefit of the teachings presented in the foregoing descriptions and the associated drawings that modifications, combinations, sub-combinations, and variations can be made without departing from the spirit or scope of this disclosure. Likewise, the various examples described may be used individually or in combination with other examples. For example, the perfusion plug can be configured so that the inlet channel extends through the perfusion plug (as in FIG. 1), and the outlet channel extends around the perfusion plug (as in FIG. 2), and vice versa. Those skilled in the art will appreciate various combinations of examples not specifically described or illustrated herein that are still within the scope of this disclosure. In this respect, it is to be understood that the disclosure is not limited to the specific examples set forth and the examples of the disclosure are intended to be illustrative, not limiting.

It is noted that the drawings may illustrate, and the description and claims may use geometric or relational terms, such as right, left, middle, above, below, upper, lower, side (i.e., area or region), length, top, bottom, on, directly, contact, coupled, attached, linear, elongated, parallel, orthogonal, distal, proximal, cylindrical, circular, etc. These terms are not intended to limit the disclosure and, in general, are used for convenience to facilitate the description based on the examples shown in the figures. In addition, the geometric or relational terms may not be exact. For instance, walls may not be exactly perpendicular or parallel to one another because of, for example, roughness of surfaces, tolerances allowed in manufacturing, etc., but may still be considered to be perpendicular or parallel. In addition, while the disclosure has been shown an described with respect to a liquid (media), it can be utilized with a gas or gel.

The foregoing description and drawings should be considered as illustrative only of the principles of the disclosure, which may be configured in a variety of shapes and sizes and is not intended to be limited by the embodiment herein described. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

1. A reactor for use with a media and a standard microtiter well plate having a plurality of wells with a side wall, a bottom portion, and a transparent bottom, and a target substance at the bottom of the well, the reactor comprising:
a support layer;
a plurality of perfusion plugs each extending from said support layer into a respective one of the plurality of wells, said perfusion plug having a distal end that is at a desired distance from the bottom of the well to form a bottom flow channel at the bottom portion of the well;
an inlet channel extending through or around said perfusion plug, said inlet channel in fluid communication with the bottom flow channel; and
an outlet channel extending through or around said perfusion plug, said outlet channel in fluid communication with the bottom flow channel,
wherein the media can travel in the inlet channel to the bottom flow channel to contact the target substance, then travel in the outlet channel to exit the reactor.

2. The reactor of claim 1, wherein said inlet channel extends around said perfusion plug, between said perfusion plug and the side wall of the well from a top portion of the well to the bottom portion of the well.

3. The reactor of claim 1, wherein said outlet channel extends around said perfusion plug, between said perfusion plug and the side wall of the well from a top portion of the well to the bottom portion of the well.

4. The reactor of claim 1, further comprising a gas and/or liquid impermeable gasket between the well and said support layer to provide a seal therebetween that is impermeable to gas and/or liquid.

5. The reactor of claim 1, wherein the support layer and perfusion plug are made of a hard acrylic.

6. The reactor of claim 1, wherein said inlet channel extends through said perfusion plug.

7. The reactor of claim 1, wherein said outlet channel extends through said perfusion plug.

8. The reactor of claim 1, further comprising an inlet connector coupled to said inlet channel.

9. The reactor of claim 1, further comprising an outlet connector coupled to said outlet channel.

10. The reactor of claim 9, further comprising a pump and a first tube connecting said pump to said inlet connector or said outlet connector, said pump transporting the media through the inlet channel to the bottom flow channel to contact the target substance, then through the outlet channel to exit the reactor at the outlet connector and the outlet tube.

11. The reactor of claim 8, further comprising a media reservoir and an media tube connecting said media reservoir to said inlet connector, said media reservoir supplying media to the inlet connector.

12. The reactor of claim 1, further comprising a temperature sensor extending along or through said support layer and said perfusion plug and in direct contact with the media in said inlet channel, said outlet channel or said bottom channel.

13. The reactor of claim 12, wherein said temperature sensor is positioned at a distal end of said perfusion plug.

14. The reactor of claim 1, further comprising a fluorescence-based oxygen sensor positioned at the well bottom, and a camera positioned to detect fluorescence of said oxygen sensor through the transparent well bottom, wherein said oxygen sensor does not interfere with optical imaging through the transparent well bottom.

15. The reactor of claim 14, wherein the oxygen sensor comprises a half-moon shape.

16. The reactor of claim 1, further comprising a flow rate sensor in flow communication with said inlet channel or said outlet channel, said flow rate sensor configured to detect a flow rate of the media in the reactor.

17. The reactor of claim 1, further comprising a controller detecting the flow rate, temperature and/or oxygen concentration, and transmitting a control signal to dynamically adjust the flow rate, temperature and/or oxygen concentration in real time.

18. The reactor of claim 17, further comprising a heater and/or cooler configured to receive the control signal and adjust the temperature of the media in response to the control signal.

19. The reactor of claim 17, further comprising a pump configured to receive the control signal and adjust the flow rate of the media in response to the control signal.

20. The reactor of claim 17, further comprising an oxygen source configured to receive the control signal and adjust the oxygen concentration of the media in response to the control signal.

21. The reactor of claim 1, further comprising a top layer extending over the support layer, said inlet channel and said outlet channel extending through the top layer.

22. The reactor of claim 1, said perfusion plug having a shape that is the same as a shape of the well.

23. The reactor of claim 1, the well plate having a plurality of wells, said reactor further comprising a plurality of perfusion plugs extending outward from the lower surface of said support layer, and a plurality of bridge flow channels each coupling adjacent ones of said plurality of perfusion plugs.

24. The reactor of claim 23, wherein said plurality of perfusion plugs are arranged in rows and columns, and said plurality of bridge flow channels couple said plurality of perfusion plugs in series along one of said rows or columns.

25. The reactor of claim 23, wherein said plurality of perfusion plugs are arranged in rows and columns, and said plurality of bridge flow channels couple said plurality of perfusion plugs in parallel.

26. The reactor of claim 23, further comprising a plurality of inlet branch channels, each having a different concentration of a reagent, gas or substance and connected to one of said plurality of perfusion plugs.

27. The reactor of claim 1, the microtiter well plate having a top surface, said support layer having a lower surface that extends substantially parallel to the top surface of the well plate, and said perfusion plug extending outward from the lower surface of said support layer.

28. The reactor of claim 1, further comprising a pump and a media reservoir coupled to the inlet channel and/or outlet channel forming a closed loop, and further comprising a controller that automatically and dynamically regulates temperature, gas concentration, and flow rate of the media in real time.

29. A method for operating a reactor having a media circulating in a plurality of wells, the method comprising:
detecting with a temperature sensor, a temperature in the plurality of wells;
adjusting by a processing device, the temperature based on the detected temperature;
detecting with a flow rate sensor, a flow rate in the plurality of wells;
adjusting by the processing device, the flow rate based on the detected flow rate;
detecting with a light detector, a fluorescence of a fluorescent-based oxygen sensor positioned in each of the plurality of wells;
determining at a processing device, a gas concentration based on the detected fluorescence;
adjusting by the processing device, the flow rate and/or gas concentration based on the determined gas concentration.
